(19) United States
(12) Patent Application Publication
(10) Pub. No.: US 2006/0173399 A1
(43) Pub. Date: Aug. 3, 2006

(54) MEMS flow module with pivoting-type baffle

(76) Inventors: M. Steven Rodgers, Albuquerque, NM (US); Norman F. Smith, Albuquerque, NM (US); Jeffry J. Sniegowski, Tijeras, NM (US); Stephen M. Barnes, Albuquerque, NM (US); Paul J. McWhorter, Albuquerque, NM (US)

Correspondence Address: MARSH, FISCHMANN & BREYFOGLE LLP, 3151 SOUTH VAUGHN WAY, SUITE 411, AURORA, CO, 80014, US

(21) Appl. No.: 11/048,195

(22) Filed: Feb. 1, 2005

(51) Int. Cl. A61F 9/007

(52) U.S. Cl. 604/9; 251/336

(57) ABSTRACT

Various embodiments of MEMS flow modules that regulate flow or pressure by the pivoting or pivoting-like movement of a flow regulating or controlling structure are disclosed. One such MEMS flow module (40) has a flow regulating structure (62) including a plurality of baffles (66) and a flow plate (50) including a plurality of flow ports (52). The flow regulating structure (62) also has a support (64) that is spaced from and anchored to the flow plate (50). Each baffle (66) is aligned with at least one flow port (52) and is interconnected to the support (64) of the flow regulating structure (62) in a manner that allows the baffles (66) to flex away from the flow plate (50) based upon the development of at least a certain differential pressure across the MEMS flow module (40).

### FIELD OF THE INVENTION

The present invention generally relates to the field of microfabricated devices and, more particularly, to a flow or pressure regulating MEMS flow module that uses at least one baffle that uses a pivotal or pivotal-like motion to provide at least a flow or pressure regulation function.

### BACKGROUND OF THE INVENTION

High internal pressure within the eye can damage the optic nerve and lead to blindness. There are two primary chambers in the eye—an anterior chamber and a posterior chamber that are generally separated by a lens. Aqueous humor exists within the anterior chamber, while vitreous humor exists in the posterior chamber. Generally, an increase in the internal pressure within the eye is caused by more fluid being generated within the eye than is being discharged by the eye. The general consensus is that it is the fluid within the anterior chamber of the eye that is the main contributor to an elevated intraocular pressure.

One proposed solution to addressing high internal pressure within the eye is to install an implant. Implants are typically directed through a wall of the patient's eye so as to fluidly connect the anterior chamber with an exterior location on the eye. There are a number of issues with implants of this type. One is the ability of the implant to respond to changes in the internal pressure within the eye in a manner that reduces the potential for damaging the optic nerve. Another is the ability of the implant to reduce the potential for bacteria and the like passing through the implant and into the interior of the patient's eye.

### BRIEF SUMMARY OF THE INVENTION

A first aspect of the present invention is embodied by a MEMS flow module. This MEMS flow module includes a first plate having a first flow port and what may be characterized as a regulating structure. The regulating structure includes a first portion that is disposed in a fixed positional relationship relative to the first plate and a second portion that at least partially extends over the first flow port. The second portion of the regulating structure flexes relative to the first portion of the regulating structure to allow for an increased flow through the MEMS flow module in a first direction (e.g., a flow through the first flow port at least generally toward the second portion of the regulating structure).

Various refinements exist of the features noted in relation to first aspect of the present invention. Further features may also be incorporated in the first aspect of the present invention as well. These refinements and additional features may exist individually or in any combination. In one embodiment, movement of the second portion of the regulating structure provides pressure and/or flow regulation capabilities. For instance, upon reaching at least a certain differential pressure, the second portion of the regulating structure may flex to increase its spacing from the first plate to allow an increased flow through the MEMS flow module. Although this “certain” differential pressure may be of any appropriate magnitude, flexing preferably starts anytime the differential pressure is greater than zero, and furthermore preferably the second portion will move anytime there is a change in the differential pressure. In another embodiment, the MEMS flow module provides a flow or pressure regulation for a flow passing through the first flow port in a first direction, and acts at least similar to a check valve by at least generally restricting or impeding a flow through the MEMS flow module in a second direction that is opposite to the noted first direction. Consider the case where the MEMS flow module is used in an implant to relieve intraocular pressure in a patient's eye, and where the MEMS flow module is disposed in a flow path between the anterior chamber of the patient's eye and another “drainage” location (e.g., exteriorly of the eye; another location within the eye or body). The MEMS flow module may be used to regulate the flow of fluid out of the anterior chamber of the patient's eye in a manner that regulates the pressure in the anterior chamber in a desired manner, and may at least substantially restrict or impede a flow from the drainage location back through the MEMS flow module and into this anterior chamber. The MEMS flow module may be designed for a laminar flow therethrough in this and other instances, although the MEMS flow module may be applicable to a turbulent flow therethrough as well.

The first portion of the regulating structure may be maintained in a fixed positional relationship with the first plate in any appropriate manner. In one embodiment, at least one anchor extends between the first portion of the regulating structure and the first plate to fixedly interconnect the first portion of the regulating structure directly to the first plate. In such an embodiment, the regulating structure and the first plate may be formed from adjacent or at least different structural MEMS layers. Further, such anchors may be utilized to interconnect the regulating structure and first plate in a parallel and/or spaced relationship.

The regulating structure may be of any appropriate size and/or shape. Preferably, the second portion of the regulating structure flexes to change the flow characteristics of the MEMS flow module in response to at least certain changes in the differential pressure across the MEMS flow module. In one embodiment, the second portion of the regulating structure may be substantially parallel to the first plate when there is no differential pressure across this second portion of the regulating structure or when the differential pressure is less than a certain amount. This may be defined as an undeformed or “zero stress” state, but in any case a “home” or first position. Other types of first positions may be appropriate, such as where there the second portion of the regulating structure is initially in contact with the first plate (e.g., by being biased into contact with the first plate). In any case, the development of at least a certain pressure differential across the MEMS flow module may flex the second portion of the regulating structure away from the first plate to a second position. The MEMS flow module could be configured to have any desired “set point” in relation to the magnitude of the differential pressure that will cause the second portion of the regulating structure to start to flex (including where this set point is zero, such that flexing will occur in response to any differential pressure greater than zero or when there is any change in the differential pressure for that matter), and thereby increase its spacing from the first plate.

Having the second portion of the regulating structure in the above-noted second position allows more flow through the MEMS flow module. The amount that the second portion of the regulating structure may flex away from the first plate may be limited or controlled in any appropriate manner (e.g., to provide a maximum spacing between the first plate and the second portion of the regulating structure when in its second position). As the magnitude of the noted pressure differential is reduced, the second portion of the regulating structure will move back at least toward its first position (e.g., using the elastic or spring forces that were created and stored within the second portion by flexing away from the first plate). That is, the internal stresses caused by flexing of the second portion of the regulating structure away from the first plate may provide a restoring force that may at least contribute to moving the second portion of the regulating structure back toward or all the way back to its first position.

In a further embodiment, the regulating structure may be an elongated member having a first portion (e.g., a first end) disposed in a fixed positional relationship relative to the first plate and at least one free end. In such an embodiment, the second portion of the regulating structure may be disposed along the length of the elongated member so as to be spaced from the fixed end (e.g., somewhere between the fixed end and the free end, or at the free end). Accordingly, the development of at least a certain pressure differential across the MEMS flow module may move the free end of the elongated member along an at least generally arcuate path and at least generally away from the first plate to accommodate an increased flow through the first flow port in the first direction. That is, the differential pressure being experienced by the second portion of the regulating structure may move the elongated member away from the first plate and the first flow port such that at least part of a flow path through the MEMS flow module is opened and/or expanded. This of course allows for an increased flow/flow rate through the MEMS flow module.

In one embodiment, the size and shape of the second portion of the regulating structure is such that it substantially covers the first flow port when the second portion of the regulating structure is disposed in a substantially adjacent relationship thereto. Accordingly, the second portion of the regulating structure may substantially restrict or impede a flow past the second portion of the regulating structure and through the first flow port in a second direction that is opposite to the first direction. A flow in the second direction through the MEMS flow module could actually move the second portion of the regulating structure into contact with the first plate and further restrict a flow through the first flow port in the second direction.

As noted above, at least a certain increase in the differential pressure across the MEMS flow module has the effect of flexing the second portion relative to the fixed first portion of the regulating structure. In one instance, this flexure allows the second portion of the regulating structure to move at least generally away from the first flow port. In any case, the position of the second portion of the regulating structure relative to the first plate will at least partially determine the flow rate through the MEMS flow module. Generally, the flow rate through the MEMS flow module will increase as the spacing between the second portion of the regulating structure and the first flow port increases, and will decrease as the spacing between these same components decreases. Preferably, the flow rate through the MEMS flow module will increase greater than proportionally for a corresponding increase in the pressure differential across the MEMS flow module.

The above-noted movement of the second portion of the regulating structure in response to the development of at least a certain pressure differential across the MEMS flow module is itself subject to a number of characterizations. One is that the second portion at least generally pivots about the fixed first portion of the structure. Another is that the second portion travels at least generally along an arcuate path either toward or away from the first plate. Yet another is that the second portion of the regulating structure may be operative to move in either of two general directions. For instance, the second portion of the regulating structure may flex so as to move at least generally away from the first flow port, which may allow for increasing the volume of a flow channel associated with the first flow port. This would then accommodate an increased flow rate through the MEMS flow module. The second portion of the regulating structure may also move at least generally toward the first flow port, which may allow for reducing the volume of this same flow channel and/or substantially restricting or impeding a flow through the first flow port in the second direction that is opposite to the first direction.

Another characterization is that the MEMS flow module may be configured such that a flow path through the MEMS flow module is always present (e.g., the MEMS flow module may be configured so as to allow at least a certain flow therethrough at all times). For instance, the second portion of the regulating structure may be spaced relative to the first flow port such that a flow path segment having at least a predetermined minimum size may be constantly maintained for receiving a flow from the first flow port in the first direction or directing a flow into and through the first flow port in the second direction. Such a flow path segment may remain open in the absence of a differential pressure adequate to flex the second portion of the regulating structure toward the first plate. Alternatively, an appropriate travel limiter or the like could be utilized to maintain a certain minimum spacing between the second portion of the regulating structure and the first plate such that the noted flow path segment always remains open. Another previously noted option would be for the second portion of the regulating structure to actually be in contact with the first plate until at least a certain differential pressure exists across the second portion of the regulating structure to move the second portion away from the first plate, to thereby open the noted flow path segment. That is, the home position for the second portion of the regulating structure could be where it contacts the first plate.

The first flow port may be of any appropriate size and/or shape. Further, the first plate may include a plurality of first flow ports that pass through the first plate. Likewise, the MEMS flow module may include a regulating structure having a plurality of second portions that at least partially extend over at least one of the plurality of first flow ports. Alternatively, the MEMS flow module may include a plurality of separate regulating structures. Such separate regulating structures each may include a first portion that is maintained in a fixed positional relationship relative to the first plate, and a second portion that at least partially extends over one or more of the plurality of first flow ports and that is operative to flex relative to the fixed first portion. In one embodiment, each regulating structure may have a first portion that is fixed relative to the first plate and a second portion that is aligned with a center of its corresponding first flow port. These first and second portions may be disposed at least generally the same distance from a common point of the MEMS flow module, for example, a geometric center of the MEMS flow module in the lateral dimension (e.g., “lateral” being in a direction that is perpendicular to a flow through the first flow port).

One or more of the first flows port may further include any appropriate structure that provides a desired flow restriction. For instance, a raised or protruding annular flow-restricting structure or wall may be disposed about a perimeter of the first flow port. This annular flow-restricting wall may be disposed between the first plate and the second portion of the regulating structure in its home position to reduce the size of a gap through which a flow must pass after passing through the first port in the first direction or prior to passing through the first flow port in the noted second direction. This annular flow-restricting wall may extend from either the first plate or the second portion of the regulating structure. In either case, the annular flow-restricting wall may limit flow through the first flow port when the second portion of the regulating structure is disposed generally adjacent thereto. Another option would be for the second portion of the regulating structure to include an appropriately shaped plug that is aligned with the first flow port. Such a plug could be disposed adjacent to one end of the first flow port (e.g., in overlying relation), or could extend within the first flow port. Preferably, any portion of such a plug that is disposed within its corresponding first flow port would be spaced from a sidewall of the first plate that defines this first flow port. The above-noted annular flow-restricting wall could also be replaced by a plurality of separate flow-restricting segments that are appropriately spaced from each other. Any way of providing a controlled flow restriction between the second portion of the regulating structure and any corresponding first flow port may be utilized. These separate flow-restricting segments would also reduce the size of the gap between the first plate and the second portion of the regulating structure in its home position. In one embodiment, the height of this gap is no more than about 0.3 microns at the noted time, although a gap of about 0.1 microns or less may be desirable in at least certain instances. Another option would be for this gap to not exist at all when the second portion of the regulating structure is in its home position.

A second aspect of the present invention is embodied by a MEMS flow module that utilizes at least one cantilever structure for providing at least a flow or pressure regulation function. The MEMS flow module includes a first plate having a first flow port and a cantilever structure. The cantilever structure includes a first portion that is fixed relative to the first plate and a free end that is operative to move along an at least generally arcuate path in response to the cantilever structure experiencing at least a certain differential pressure.

Various refinements exist of the features noted in relation to the second aspect of the present invention. Further features may also be incorporated in the second aspect of the invention as well. These refinements and additional features may exist individually or in any combination. Although the “certain” differential pressure may be of any appropriate magnitude, the free end of the cantilever preferably moves anytime the differential pressure is greater than zero or anytime there is a change in the differential pressure. The MEMS flow module may also include a second plate having a second flow port. The second plate may be formed directly on or disposed in interfacing relation with the first plate, with the first and second flow ports being fluidly interconnected in any appropriate manner. Another option would be for this second plate to be spaced from and fixedly interconnected with the first plate in any appropriate manner. For instance, such first and second plates may be fixedly interconnected such that the flow ports through these plates are at least partially aligned, although such is not required. Furthermore, to reduce the potential for fluid flow out from between the first and second plates, one or more annular seals may be utilized to interconnect the first and second plates around the perimeter of the regions of the first and second plates having the first and second flow ports.

The cantilever structure may be of any appropriate size and/or shape. For instance, the cantilever structure may be formed as a simple beam that extends between the fixed first portion and the free end. What is important is that a portion of the cantilever structure between the fixed first portion and the free end be disposed at least generally adjacent to the first flow port in the absence of at least a certain differential pressure (thereby including where the differential pressure is zero). Accordingly, when at least a certain pressure differential develops across the MEMS flow module, a force is exerted on the cantilever structure to accommodate an increased flow through the first flow port. This differential pressure may be operative to move the cantilever structure away from the first flow port and thereby define or increase at least a segment of a flow path through the MEMS flow module. Preferably, the volume of this flow path segment, and thereby a flow rate through this flow path segment, will increase greater than proportionally for a corresponding increase in the differential pressure across the MEMS flow module.

In one embodiment, the first plate may include a plurality of first flow ports. Accordingly, a cantilever structure may be utilized that has a single fixed portion and a corresponding plurality of free ends. For instance, a plurality of separate beams may extend from a single fixed portion or a single beam may extend from a single fixed portion and may include a plurality of branching segments, each having a free end (e.g., a “Y”-shaped configuration). Alternatively, a plurality of separate cantilever structures may be utilized, where each cantilever structure includes a first portion that is fixed relative to the first plate and at least one free end.

A third aspect of the present invention is embodied by a MEMS flow module. The MEMS flow module includes a flow regulator that includes at least one movable baffle, and more preferably a plurality of independently movable baffles. The MEMS flow module further includes a first plate, which in turn includes at least one first flow port, and more preferably a plurality of first flow ports. Each baffle may be aligned with its own first flow port or group of first flow ports. It may be possible for multiple baffles to interact with a common first flow port as well. In any case, at least one baffle (including all baffles) at least generally pivots or undergoes a pivotal-like motion to change a magnitude of spacing of the baffle from the first plate in response to at least a certain change in a differential pressure across the MEMS flow module.

Various refinements exist of the features noted in relation to the third aspect of the present invention. Further features may also be incorporated into the third aspect of the present invention as well. These refinements and additional features may exist individually or in any combination. Although the amount of differential pressure required to move the baffle(s) may be of any appropriate value, preferably the baffle(s) moves anytime the differential pressure is greater than zero or anytime there is a change in the differential pressure.

The flow regulator may be of any appropriate configuration that is operative to control the flow through the various first flow ports used by the first plate. In one embodiment, each baffle undergoes a pivotal or pivotal-like motion (e.g., each baffle moves at least generally about a certain axis). Any configuration that allows for or accommodates this general type of movement may be utilized in relation to the third aspect. For instance, a particular baffle could flex or have its free end move at least generally along an arcuate path in the manner noted above in relation to the first and second aspects to provide the movement contemplated by this third aspect. Another option would be for a particular baffle to move in the manner contemplated by this third aspect by using one or more appropriate hinges or hinge-like structures. Yet another option would be for a particular baffle to move in the manner contemplated by this third aspect by using a torsional deflection (e.g., by having a baffle extend from or otherwise be interconnected with a beam or other structure that torsionally deflects to allow each baffle mounted thereon to move in the desired manner).

In one embodiment, the flow regulator includes a common support to which a plurality of baffles are interconnected. This support may be spaced from and structurally interconnected with the first plate in any appropriate manner (e.g., by a plurality of posts or columns extending therebetween). The first plate could also directly interface with this support. In one embodiment, the support includes a perimeter, wherein an entirety of a region inward of the perimeter is occupied by the support, except possibly for any etch release holes that may extend through the support as will be discussed in more detail below. The plurality of baffles may be interconnected with this support, such as about its perimeter. In another embodiment, the support includes an aperture or cutout section, as well as an annular section that is disposed about this aperture and from which the plurality of baffles extend. In this embodiment, the annular section may be structurally interconnected with the first plate and the plurality of baffles may be interconnected with, for example, an outside perimeter of the annular section. Generally, each of the plurality of baffles may extend directly from the support and be operative to flex relative to the first plate. In one particular arrangement, each of the plurality of baffles extends along separate radii that emanate from a common point or location (e.g., a plurality of baffles could extend radially outwardly from a common central support; a plurality of baffles could extend radially inwardly from a common support). Notwithstanding the foregoing, a plurality of baffles may be disposed in any desired/required arrangement.

One or more structures may be incorporated in order to reduce the size of a flow path between each of the baffles and the first plate. An annular flow-restricting wall (“annular”, as used herein, means extending a full 360 degrees about a common point, and thereby does not require a circular configuration) may be associated with each first flow port or first flow port group, and may be disposed within part of the space between the first plate and the corresponding baffle. Such an annular flow-restricting wall may be used to reduce the size of a space through which a flow must pass after proceeding through a first flow port in the direction of the corresponding baffle. In one embodiment, the height of this space is no more than about 0.3 microns when there is no differential pressure across the corresponding baffle, although a gap of about 0.1 microns or less may be desirable in at least certain instances. As noted above, there may be no space at all when there is no differential pressure across the corresponding baffle (e.g., the baffle may be in contact with the annular flow-restricting wall).

Depending upon how the MEMS flow module is fabricated, an annular flow-restricting wall of the above-noted type may extend from the first plate and terminate prior to reaching the corresponding baffle, or such an annular flow-restricting wall may extend from the corresponding baffle and terminate prior to reaching the first plate. A plurality of flow-restricting wall segments that are appropriately spaced could be used in place of an annular flow-restricting wall as well. Another option would be for a particular baffle to include an appropriate plug that is aligned with its corresponding first flow port. Such a plug could be disposed adjacent to one end of its corresponding first flow port (e.g., in closely overlying relation), or could actually extend within the first flow port as noted above in relation to the first aspect. These types of plugs also provide a flow restriction that may be a benefit, for instance by providing only a small space between the perimeter of the plug and a wall of the first plate that defines its corresponding first flow port.

The MEMS flow device may include a second plate that includes a plurality of second flow ports, and that is disposed such that the first plate is located somewhere between the flow regulator and the second plate. Each of these second flow ports may be aligned with a corresponding first flow port through the first plate, although such is not required (e.g. an offset relationship could exist between each second flow port(s) and its corresponding first flow port(s)). Further, the MEMS flow module may include one or more structural connections extending between the first and second plates to maintain the same in spaced relation (e.g., parallel). Utilization of the first and second plates and an appropriate number of structural connections therebetween may allow for increasing the overall stiffness of the MEMS flow module. Furthermore, the structural connections may include one or more annular connections that are disposed about any corresponding pairs of first and second flow ports. Further in this regard, one or more annular supports may be disposed beyond the perimeter of the free ends of the various baffles and also may structurally interconnect the first and second plate. This may also provide one or more radial seals as noted above. Yet another option would be to fabricate the MEMS flow module such that the second plate is actually disposed directly on the first plate. An appropriate fluid interconnection would of course be required between a particular second flow port and its corresponding first flow port(s).

The MEMS flow module may the fabricated such that at least certain portions thereof require the use of a plurality of etch release holes (e.g., about 1 micron or less diameter holes than extend through the relevant structure). Such etch release holes may extend through one or more of the first plate, the baffles, and any second plate to allow an etchant to remove a sacrificial material during what is commonly referred to as a “release.” The MEMS flow module may further include a flow restrictor for one or more of these etch release holes, and including for each such etch release hole. Such flow restrictors may be configured to provide a desirable flow rate (e.g., a low or limited flow rate) through any associated etch release hole. One or more of the flow restrictors may also provide a filtering function, which may be desirable for one or more applications.

A plurality of etch release holes may extend through the first plate, while a plurality of etch release holes may extend through the various baffles. A flow restrictor may be provided for one or more of the etch release holes through the first plate. It may be such that a flow restrictor is only required for those etch release holes through the baffles that are aligned with or directly exposed by a flow through a first flow port of the first plate. Generally, a separate flow restrictor may be provided for any number of etch release holes through the first plate and for any number of etch release holes through the various baffles to provide the desired flow restriction. It also may be possible to fabricate the MEMS flow module without having to use any etch release holes through either the first plate or the various baffles (e.g., using etch release rails in one or more underlying fabrication levels).

A plurality of baffles may extend from a common support in a spoke-like fashion. For instance, a plurality of baffles could extend outwardly from a common central support, or a plurality of baffles could extend inwardly from a common outer support (e.g., an outer annular ring). Another option would be for a plurality of baffles to be symmetrically disposed about a reference point that is within an area or region whose perimeter is in effect collectively defined by the various baffles. Corresponding portions of the plurality of baffles in this second instance may be disposed at least substantially the same distance from the noted reference point, and each such baffle may include a first point that is aligned with a center of its corresponding first flow port and a second point that corresponds with a center that is associated with where the baffle is anchored. The first and second points of each baffle may be disposed at generally the same distance from the noted reference point. This type of arrangement for the plurality of baffles may reduce the effects of a flexing or deflection of the first plate when exposed to a certain differential pressure. Notwithstanding the foregoing, a plurality of baffles may be disposed in any appropriate arrangement in relation to the third aspect.

Surface micromachining is the preferred technology for fabricating the MEMS flow modules of the first, second, and third aspects. In this regard, these MEMS flow modules may be fabricated in at least two different fabrication levels that are spaced from each other (hereafter a first fabrication level and a second fabrication level). “Fabrication level” corresponds with what may be formed by a deposition of a structural material before having to form any overlying layer of a sacrificial material (e.g., from a single deposition of a structural layer or film). The flow controlling structure (the second portion of the flow regulating structure (first aspect), the cantilever structure (second aspect), and baffles (third aspect)) may be fabricated at least in the first fabrication level, while the first plate may be fabricated in at least the second fabrication level. It should be appreciated that the characterization of the flow controlling structure being in the “first fabrication level” and the first plate being in the “second fabrication level” by no means requires that the first fabrication level be that which is deposited “first”, and that the second fabrication level be that which is deposited “second.” Moreover, it does not require that the first fabrication level and the second fabrication level be immediately adjacent to each other. These MEMS flow modules may be fabricated on an appropriate substrate and where the first plate is fabricated in one structural layer that is disposed somewhere between the substrate and another structural layer in which the flow controlling structure is fabricated, or vice versa.

One or both of the flow controlling structure and the first plate each may exist in a single fabrication level or may exist in multiple fabrication levels. In the above-noted first instance, a deposition of a structural material in a single fabrication level may define an at least generally planar layer. Another option regarding the first instance would be for the deposition of a structural material in a single fabrication level to define an at least generally planar portion, plus one or more structures that extend down toward, but not to, the underlying structural layer at the underlying fabrication level. In either situation and prior to the release, in at least some cases there will be at least some thickness of sacrificial material disposed between the entirety of the flow controlling structure and the first plate.

In the above-noted second instance, two or more structural layers or films from adjacent fabrication levels could be disposed in direct interfacing relation (e.g., one directly on the other). Over the region that is to define the flow controlling structure or first plate, this would require removal of the sacrificial material that is deposited on the structural material at one fabrication level before depositing the structural material at the next fabrication level. Another option regarding the above-noted second instance would be to maintain the separation between structural layers or films in different fabrication levels for the flow controlling structure and/or first plate, but provide an appropriate structural interconnection therebetween (e.g., a plurality of columns, posts, or the like extending between adjacent structural layers or films in different, spaced fabrication levels).

The MEMS flow modules of the first, second, and third aspects are preferably passive devices (no external electrical signal of any type required) and may be used for any appropriate application. Another characterization of these MEMS flow modules is that they are autonomous in that they are self-contained structures and require no external power. For instance, any of these MEMS flow modules may be disposed in a flow path of any type (e.g., between a pair of sources of any appropriate type, such as a man-made reservoir, a biological reservoir, and/or the environment), and further may be used for any appropriate application. That is, one or more of any of these MEMS flow modules could be disposed in a conduit that fluidly interconnects multiple sources (e.g., two or more), and each source may be either a man-made reservoir, a biological reservoir, the environment, or any other appropriate source. One example would be to dispose one or more of these MEMS flow modules in a conduit extending between the anterior chamber of an eye and a location that is exterior of the cornea of the eye. Another example would be to dispose one or more of these MEMS flow modules in a conduit extending between the anterior chamber of an eye and another location that is exterior of the sclera of the eye. Yet another example would be to dispose one or more of these MEMS flow modules in a conduit extending between the anterior chamber of an eye and another location within the eye (e.g., into Schlemm's canal) or body. In any case, any of these MEMS flow modules could be disposed directly into such a conduit, or one or more housings could be used to integrate any of these MEMS flow modules with the conduit.

### BRIEF DESCRIPTION OF THE SEVERAL VIEWS OF THE DRAWING

FIG. 1 is a side view of a plurality of layers that may be used by one embodiment of a surface micromachining fabrication technique.

FIG. 2A is a perspective view of a first embodiment of a MEMS flow module.

FIG. 2B is a cross-sectional, exploded, perspective view of the MEMS flow module of FIG. 2A.

FIG. 2C is a cross-sectional view through one of the baffles of the MEMS flow module of FIG. 2A.

FIGS. 2D and 2E are cross-sectional views of representative flow restrictors for etch release holes that may be used by any of the MEMS flow modules described herein.

FIG. 3A is a perspective view of a second embodiment of a MEMS flow module.

FIG. 3B is a cross-sectional, exploded, perspective view of the MEMS flow module of FIG. 3A.

FIG. 3C is a perspective view of release rails that may be utilized in the fabrication of the MEMS flow module of FIG. 3A.

FIG. 4A is a perspective view of a third embodiment of a MEMS flow module.

FIG. 4B is a cross-sectional, exploded, first partial perspective view of the MEMS flow module of FIG. 4A.

FIG. 4C is a cross-sectional, exploded, second perspective view of the MEMS flow module of FIG. 4A.

FIG. 5A is a perspective view of a fourth embodiment of a MEMS flow module.

FIG. 5B is a partially exploded, perspective view of the opposite side of the MEMS flow module illustrated in FIG. 5A.

FIG. 6 is a cross-sectional view of a baffle with a flow-controlling plug that may be utilized with any of the MEMS flow modules of FIGS. 2A-5B.

FIG. 7 is an exploded, perspective view of one embodiment of a flow assembly that uses a MEMS flow module.

FIG. 8 is a perspective view of the flow assembly of FIG. 7 in an assembled condition.

FIG. 9A is an exploded, perspective of another embodiment of a flow assembly that uses a MEMS flow module.

FIG. 9B is a perspective view of the flow assembly of FIG. 9A in an assembled condition.

FIG. 10A is an exploded, perspective of another embodiment of a flow assembly that uses a MEMS flow module.

FIG. 10B is a perspective view of the flow assembly of FIG. 10A in an assembled condition.

FIG. 11A is a schematic of one embodiment of an implant that may use any of the MEMS flow modules described herein.

FIG. 11B is a cross-sectional view of one embodiment of an implant or shunt that is used to relieve pressure within the anterior chamber of the eye, and that may utilize any of the MEMS flow modules described herein.

### DETAILED DESCRIPTION OF THE INVENTION

The present invention will now be described in relation to the accompanying drawings that at least assist in illustrating its various pertinent features. Generally, the devices described herein are microfabricated. There are a number of microfabrication technologies that are commonly characterized as “micromachining,” including without limitation LIGA (Lithographie, Galvonoformung, Abformung), SLIGA (sacrificial LIGA), bulk micromachining, surface micromachining, micro electrodischarge machining (EDM), laser micromachining, 3-D stereolithography, and other techniques. Hereafter, the term “MEMS device,” “microfabricated device,” or the like means any such device that is fabricated using a technology that allows realization of a feature size of 10 microns or less.

Surface micromachining is currently the preferred fabrication technique for the various devices to be described herein. One particularly desirable surface micromachining technique is described in U.S. Pat. No. 6,082,208, that issued Jul. 4, 2000, that is entitled “Method For Fabricating Five-Level Microelectromechanical Structures and Microelectromechanical Transmission Formed,” and the entire disclosure of which is incorporated by reference in its entirety herein. Surface micromachining generally entails depositing alternate layers of structural material and sacrificial material using an appropriate substrate (e.g., a silicon wafer) which functions as the foundation for the resulting microstructure. Various patterning operations (collectively including masking, etching, and mask removal operations) may be executed on one or more of these layers before the next layer is deposited so as to define the desired microstructure. After the microstructure has been defined in this general manner, all or a portion of the various sacrificial layers are removed by exposing the microstructure and the various sacrificial layers to one or more etchants. This is commonly called “releasing” the microstructure.

The term “sacrificial layer” as used herein means any layer or portion thereof of any surface micromachined microstructure that is used to fabricate the microstructure, but which does not exist in the final configuration. Exemplary materials for the sacrificial layers described herein include undoped silicon dioxide or silicon oxide, and doped silicon dioxide or silicon oxide (“doped” indicating that additional elemental materials are added to the film during or after deposition). The term “structural layer” as used herein means any other layer or portion thereof of a surface micromachined microstructure other than a sacrificial layer and a substrate on which the microstructure is being fabricated. Exemplary materials for the structural layers described herein include doped or undoped polysilicon and doped or undoped silicon. Exemplary materials for the substrates described herein include silicon. The various layers described herein may be formed/deposited by techniques such as chemical vapor deposition (CVD) and including low-pressure CVD (LPCVD), atmospheric-pressure CVD (APCVD), and plasma-enhanced CVD (PECVD), thermal oxidation processes, and physical vapor deposition (PVD) and including evaporative PVD and sputtering PVD, as examples.

In more general terms, surface micromachining can be done with any suitable system of a substrate, sacrificial film(s) or layer(s) and structural film(s) or layer(s). Many substrate materials may be used in surface micromachining operations, although the tendency is to use silicon wafers because of their ubiquitous presence and availability. The substrate is essentially a foundation on which the microstructures are fabricated. This foundation material must be stable to the processes that are being used to define the microstructure(s) and cannot adversely affect the processing of the sacrificial/structural films that are being used to define the microstructure(s). With regard to the sacrificial and structural films, the primary differentiating factor is a selectivity difference between the sacrificial and structural films to the desired/required release etchant(s). This selectivity ratio may be on the order of about 10:1, and is more preferably several hundred to one or much greater, with an infinite selectivity ratio being most preferred. Examples of such a sacrificial film/structural film system include: various silicon oxides/various forms of silicon; poly germanium/poly germanium-silicon; various polymeric films/various metal films (e.g., photoresist/aluminum); various metals/various metals (e.g., aluminum/nickel); polysilicon/silicon carbide; silicone dioxide/polysilicon (i.e., using a different release etchant like potassium hydroxide, for example). Examples of release etchants for silicon dioxide and silicon oxide sacrificial materials are typically hydrofluoric (HF) acid based (e.g., concentrated HF acid, which is actually 49 wt % HF acid and 51 wt % water; concentrated HF acid with water; buffered HF acid (HF acid and ammonium fluoride)).

The microfabrication technology described in the above-noted '208 patent uses a plurality of alternating structural layers (e.g., polysilicon and therefore referred to as “P” layers herein) and sacrificial layers (e.g., silicon dioxide, and therefore referred to as “S” layers herein). The nomenclature that is commonly used to describe the various layers in the microfabrication technology described in the above-noted '208 patent will also be used herein.

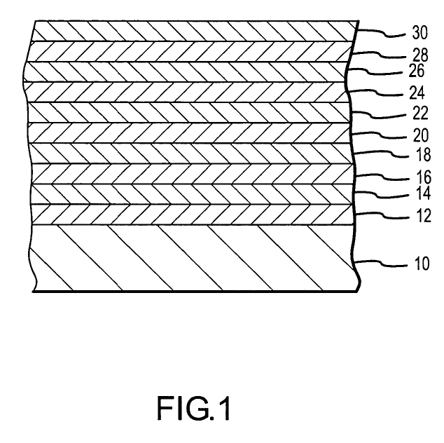

FIG. 1 generally illustrates one embodiment of layers on a substrate 10 that is appropriate for surface micromachining and in accordance with the nomenclature commonly associated with the '208 patent. Progressing away from the substrate 10, the various layers are: a dielectric layer 12 (there may be an intermediate oxide layer between the dielectric layer 12 and the substrate 10 as well, which is not shown); a P₀ layer 14 (a first fabrication level); an S₁ layer 16; a P₁ layer 18 (a second fabrication level); an S₂ layer 20; a P₂ layer 22 (a third fabrication level); an S₃ layer 24; a P₃ layer 26 (a fourth fabrication level); an S₄ layer 28; and a P₄ layer 30 (a fifth fabrication level). In some cases, the S₂ layer 20 may be removed before the release such that the P₂ layer 22 is deposited directly on the P₁ layer 18, and such may hereafter be referred to as a P₁/P₂ layer. It should also be appreciated that one or more other layers may be deposited on the P₄ layer 30 after the formation thereof and prior to the release, where the entirety of the S₁ layer 16, S₂ layer 20, S₃ layer 24, and S₄ layer 28 may be removed (although portions of one or more of these layers may be retained for one or more purposes if properly encased so as to be protected from the release etchant). It should also be appreciated that adjacent structural layers may be structurally interconnected by forming cuts or apertures through the entire thickness of a particular sacrificial layer before depositing the next structural layer. In this case, the structural material will not only be deposited on the upper surface of the particular sacrificial layer, but will be deposited in these cuts or apertures as well (and will thereby interconnect a pair of adjacent, spaced, structural layers).

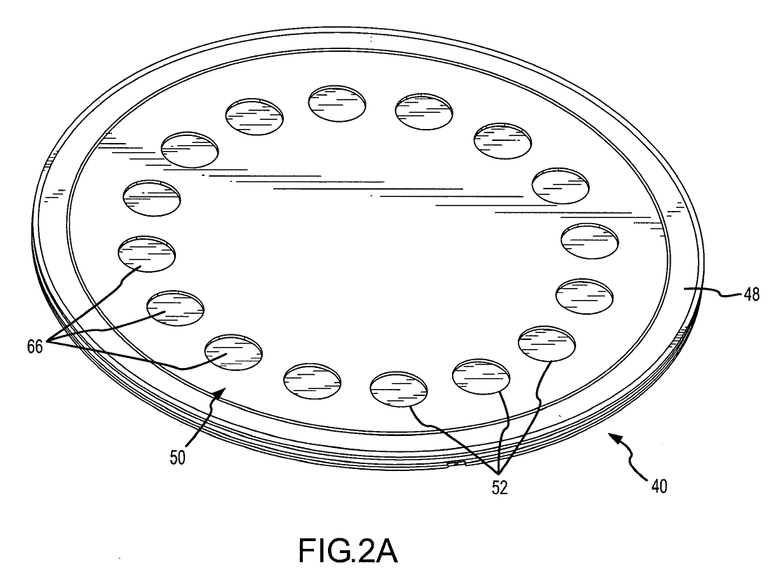

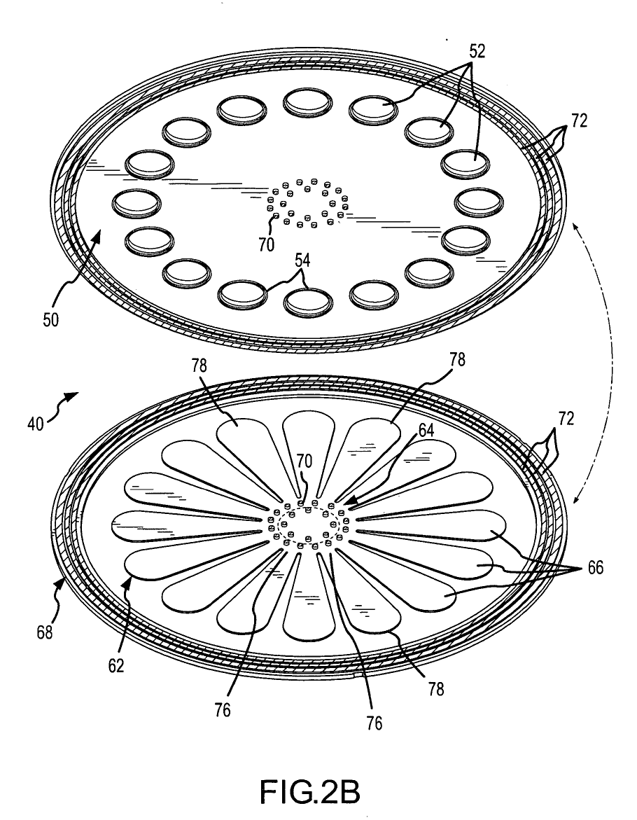

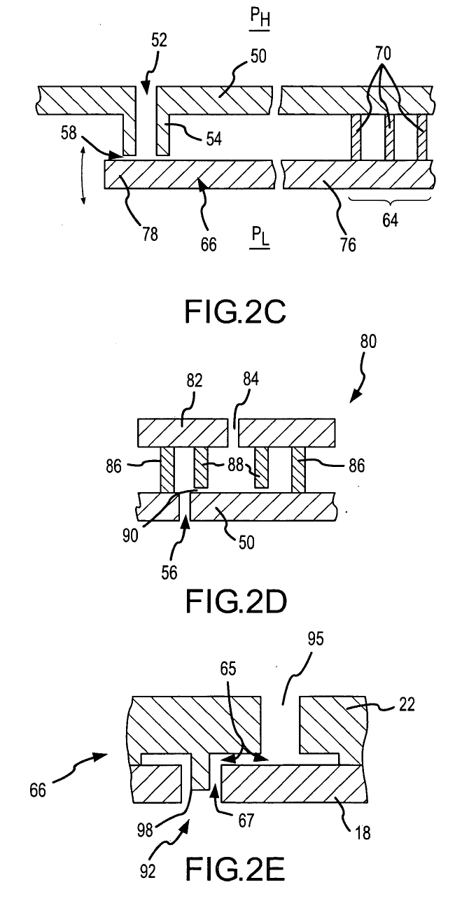

The general construction of one embodiment of a MEMS flow module (a MEMS device) is illustrated in FIGS. 2A-C, is identified by reference numeral 40, and provides pressure or flow regulation capabilities, filtration capabilities, or both. Although the MEMS flow module 40 is illustrated as having a circular configuration in plan view, any appropriate configuration may be utilized and in any appropriate size.

As shown in FIGS. 2A-2B, the MEMS flow module 40 includes a flow plate 50 (e.g., fabricated in P₃ layer 26) having a plurality of flow ports 52 that extend completely through the flow plate 50 and that are equally spaced about a common center point in the illustrated embodiment. Any number of flow ports 52 may be utilized, and the flow ports 52 may be of any appropriate size and/or configuration. The flow ports 52 could also be disposed in other appropriate arrangements. It would be typical to configure the MEMS flow module 40 (as well as the other MEMS flow modules to be described herein) to allow a target flow rate for a target differential pressure. The flow rate through the MEMS flow module 40 at other differential pressures would depend on the various characteristics of the MEMS flow module 40.

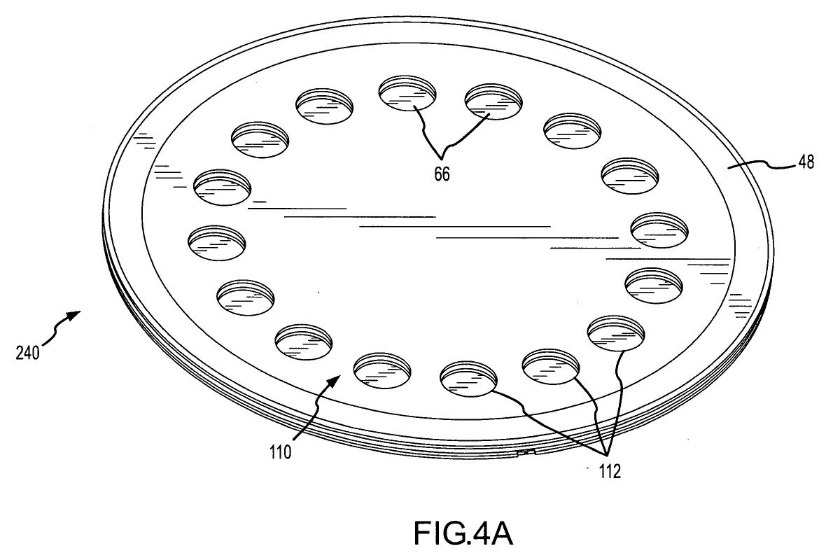

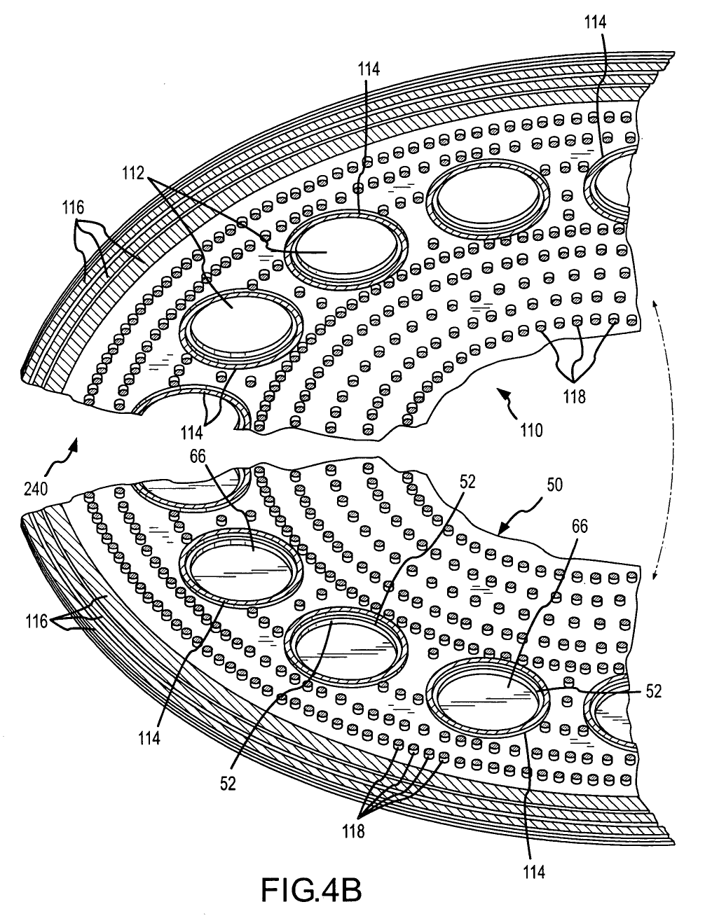

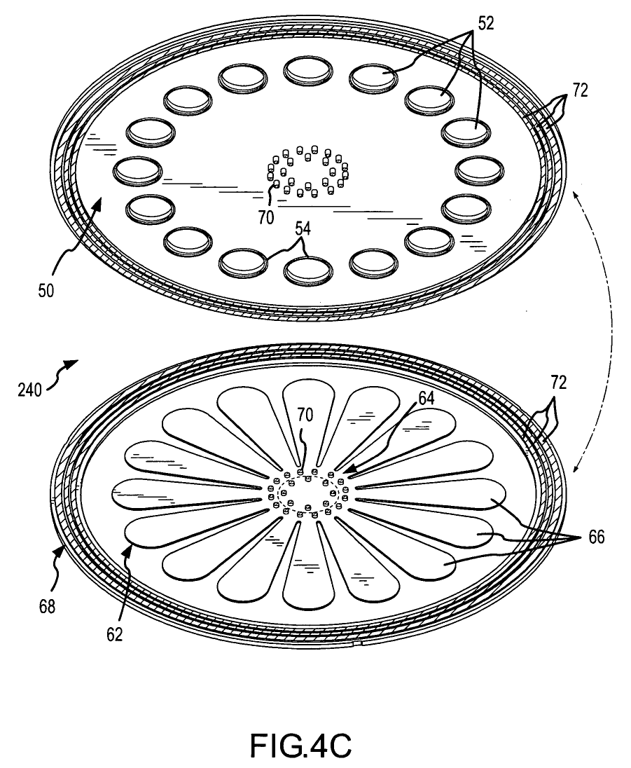

The MEMS flow module 40 further includes a flow controlling or regulating structure 62 (e.g., fabricated in the P₂ layer 22 or in a combined P₂ layer 22/P₁ layer 18) and an outer support ring 68 (e.g., fabricated in the P₂ layer 22 or in a combined P₂ layer 22/P₁ layer 18). That is, with the regulating structure 62 being in an undeformed state (e.g., where there is no differential pressure), the outer support ring 68 and the regulating structure 62 may be disposed in at least generally coplanar relation. The outer support ring 68 may be of any appropriate size, shape, and/or configuration. In the illustrated embodiment, the outer support ring 68 is annular in that it extends a full 360 degrees about a common point. “Annular” does not require the outer support ring 68 to be circular. The MEMS flow module 40 could include one or more additional flow plates that each have one or more flow ports. For instance, another such flow plate could be provided such that the regulating structure 62 is “sandwiched” between this additional flow plate and the flow plate 50. Another flow plate could be provided in the manner of the embodiment of FIGS. 4A-C. Both of these additional flow plates could be utilized as well. Any additional flow plate or flow plates could be disposed in spaced relation to another flow plate (e.g., including being fixedly interconnected therewith through one or more structural interconnections of any appropriate type) or could be disposed in interfacing relation with another flow plate (e.g., a flow plate could be fabricated in the P₄ layer 30, that in turn is deposited directly on a flow plate 50 that is fabricated in the P₃ layer 26).

The regulating structure 62 includes a center portion or support 64 and a plurality of cantilevered structures or baffles 66 that may be characterized as extending radially outwardly from the support 64 (e.g., in spoke-like fashion). It should be appreciated that the baffles 66 could extend radially inwardly from a common support as well, such as from the outer support ring 68 (not shown). That is, the support 64 provides a supporting function for the baffles 66, which cantilever from the support 64 (e.g., one end 76 of each baffle 66 is attached to the support 64, while the opposite end 78 is “free” or unsupported). Generally, both the support 64 and baffles 66 may be of any appropriate size/shape/configuration that allows each baffle 66 to flex for purposes of changing the spacing between the baffles 66 and the flow plate 50. In the illustrated embodiment, each baffle 66 flexes at least generally about an axis that is perpendicular to its length dimension (corresponding with the distance from where a particular baffle 60 attaches the support 64 and its free end 78). Removing a center portion of the support 64 (e.g., a region such as that identified by the dashed lines in FIG. 2B) of the flow regulating structure 62 may reduce the rigidity of the flow plate 50, which may be desirable for at least one or more applications. That is, removing the above-noted portion of the support 64 may allow the flow plate 50 to flex more than the configuration presented in FIG. 2B.

Any number of baffles 66 may be used, although each baffle 66 will be associated with at least one flow port 52 through the flow plate 50, and the baffles 66 may be disposed in any appropriate arrangement. In the illustrated embodiment, the baffles 66 are equally spaced about the support 64 and at least generally extend from a common location (e.g., the length dimension of each baffle 66 is disposed along a radii emanating from a common point). As shown, each baffle 66 has a free end 78 that is operable to move relative to the flow plate 50 in relation to the development of at least a certain pressure differential across the MEMS flow module 40. Further, each baffle 66 is sized to overlay (e.g., be disposed over or in overlying relation) a corresponding flow port 52 when the baffle 66 is in an adjacent relationship to the flow plate 50. Although the amount of differential pressure required to flex the baffles 66 may be of any appropriate magnitude, preferably the baffles 66 will move to at least some degree anytime the differential pressure is greater than zero or anytime there is a change in the differential pressure. Accordingly, movement of the baffles 66 relative to the flow plate 50 regulates flow through the corresponding flow ports 52. The function of the baffles 66 will be more fully discussed herein.

In the illustrated embodiment, the flow plate 50 exists in at least one fabrication level, and the regulating structure 62 exists in at least one different fabrication level (e.g., the flow plate 50 and the regulating structure 62 may be fabricated in adjacent structural layers of the MEMS device). Specifically, the flow plate 50 may be fabricated in the P₃ layer 26 and the regulating structure 62 may be fabricated in at least the P₂ layer 22 (see FIG. 1). The MEMS flow module 40 may include a ring 48 that is fixedly interconnected to the outside perimeter of the top surface of the flow plate 50 or that which is opposite the outer support ring 68. That is, an annular portion of the flow plate 50 may be “sandwiched” between the ring 48 and the outer support ring 68. This ring 48 may be a metallic ring that is attached to or formed on the flow plate 50 after the MEMS flow module 40 has been fabricated, or, may be made from another fabrication level (e.g., P₄ layer 30). Generally, the ring 48 may provide a desired interface with a housing or other structure that incorporates the MEMS flow module 40.

As will be appreciated, the various components of the MEMS flow module 40 may be formed within different layers of a MEMS structure. Furthermore, it will be appreciated that, unless otherwise stated, the various components of the MEMS flow module 40 may be formed in a MEMS structure in a reverse order as well. However, in the embodiment shown, the regulating structure 62 is formed at least in the P₂ layer 22 (also possibly in the P₁ layer 18, where the P₂ layer 22 and P₁ layer 18 are disposed in interfacing relation) and the flow plate 50 is formed in the P₃ layer 26. Accordingly, upon the removal of the S₃ layer 24 by the release in this case, a spacing of approximately 2 microns may exist between the lower surface of the flow plate 50 and each of the upper surface of the regulating structure 62 and the upper surface of the outer support ring 68.

FIG. 2B shows an exploded, perspective view of the MEMS flow module 40. Specifically, FIG. 2B is a cross-section of the MEMS flow module 40 that is taken along a plane that is parallel to the flow plate 50, at a location that is between the flow plate 50 and the regulating structure 62 in the space between a plurality of flow-restricting rings 54 (discussed below) and the regulating structure 62, and with the flow plate 50 having been rotated or pivoted away from the regulating structure 62 and outer support ring 68. As shown, various structures are formed during the microfabrication process to interconnect the regulating structure 62 to the flow plate 50, as well as to interconnect the outer support ring 68 to the bottom perimeter of the flow plate 50. More specifically, a plurality of interconnects or anchors 70 are formed between the support 64 of the regulating structure 62 and a bottom, center portion of the flow plate 50. Any number of anchors 70 may be utilized, the anchors 70 may be of any appropriate size, shape, and configuration, and the anchors 70 may be disposed in any appropriate arrangement. Likewise, a plurality of “radially spaced” annular connectors 72 are formed between the outer support ring 68 and the bottom of the flow plate 50 at a location so as to encompass all flow ports 52. “Annular” again only means that the connectors 72 extend a full 360 degrees about a common reference point, and thereby does not limit the connectors 72 to having a circular configuration. Any number of connectors 72 may be utilized. Using multiple, radially spaced connectors 72, as shown, provides redundant radial seals, which may be desirable for one or more applications.

Consider the case where the regulating structure 62 and outer support ring 68 are fabricated at least in the P₂ layer 22 (again, typically the P₂ layer 22 and P₁ layer 18 will be disposed in interfacing relation). In this case, the anchors 70 and annular connectors 72 could be fabricated after the regulating structure 62 and outer support ring 68 have been patterned from at least the P₂ layer 22. Once these structures 62, 68 have been fabricated, the S₃ layer 24 may be deposited on top of both the regulating structure 62 and outer support ring 68, as well as into the space between the individual baffles 66 and into the space between the regulating structure 62 and the outer support ring 68. The S₃ layer 24 may then be patterned to define a plurality of holes therein that extend down to the P₂ layer 22 to correspond with the desired cross-sectional configuration and location of the anchors 70, and the S₃ layer 24 may also be patterned to define a plurality of annular trenches that extend down to the P₂ layer 22 to correspond with the desired cross-sectional configuration and location of the annular connectors 72. These holes and trenches extend all the way through the S₃ layer 24 and down to the P₂ layer 22. The P₃ layer 26 may then be deposited onto the upper surface of the S₃ layer 24 and into the holes and trenches in the S₃ layer 24. This P₃ layer 26 may then be patterned to define the perimeter of the flow plate 50 and the various flow ports 52 extending therethrough. The anchors 70, annular connectors 72, and flow plate 50 are thereby fabricated from the P₃ layer 26 and exist at a common fabrication level. Accordingly, the anchors 70 fixedly interconnect the support 64 of the regulating structure 62 to the bottom surface of the flow plate 50, and the annular connectors 72 fixedly interconnect the outer support ring 68 to a bottom of the flow plate 50.

FIG. 2C illustrates the general operation of a representative flow port 52 and a corresponding baffle 66. As shown in FIG. 2C, the flow plate 50 and baffle 66 are shown in a home or first position, or, stated another way, a pressure differential across the MEMS flow module 40 is not yet sufficient to deflect the baffle 66 away from the flow plate 50 (preferably, this is the position when there is no differential pressure across the baffles 66). In the latter regard, a first pressure P_{H} above the flow plate 50 is not sufficiently greater than a second pressure P_{L} below the baffle 66 to result in deflection of the baffle 66 away from the flow plate 50. In this home position, the flow plate 50 and baffle 66 may be spaced approximately 2 microns apart in accordance with a typical spacing between adjacent structural/fabrication MEMS layers. While the pressure differential across the MEMS flow module 40 may not be sufficient to appreciably deflect the baffle 66, a pressure differential may still be present. Accordingly, if a 2 micron spacing were maintained between the baffle 66 and the flow plate 50, an undesired flow may proceed through the MEMS flow module 40 from the side of the first pressure P_{H} to the side of the second pressure P_{L}. Such an undesired flow may be addressed by providing an appropriate structure for each flow port 52 to create a flow restriction of a desired magnitude/amount. In the illustrated embodiment, a flow-restricting structure in the form of an annular flow-restricting wall or ring 54 is provided for each flow port 52. “Annular” means that the flow-restricting ring 54 extends a full 360 degrees about a common point, and does not limit the flow-restricting ring 54 to a circular configuration. Other types of flow-restricting structures could be utilized as well. For instance, each flow-restricting ring 54 could be replaced by a plurality of flow-restricting segments of any appropriate size/shape/configuration, where adjacent pairs of flow-restricting segments would be appropriately spaced from each other. The gap between such flow-restricting segments and the corresponding baffle 66, as well as the gap between adjacent pairs of flow-restricting segments, would provide the desired degree of flow restriction. A common flow-restricting structure could also be associated with a plurality of first flow ports 52 (e.g., a flow-restricting ring 54 or a plurality of flow restricting segments could be collectively disposed about a group of first flow ports 52).

In the case where the flow plate 50 is fabricated in a level that is further from the substrate 10 than the regulating structure 62, each annular flow-restricting ring 54 may be disposed on the bottom surface of the flow plate 50, or that surface which faces the regulating structure 62. In the case where the flow plate 50 is fabricated in a level that is closer to the substrate 10 than the regulating structure 62, each annular flow-restricting ring 54 may be disposed on the upper surface a baffle 66, or that surface which faces the flow plate 50. In either case, the function of each flow-restricting ring 54 is to reduce the size of a flow channel between the associated baffle 66 and flow port 52. In one embodiment and with the baffles 66 in an un-deflected state or in the “home” position of FIG. 2C, a gap 58 between the bottom of the flow-restricting ring 54 and its corresponding baffle 66 in the illustrated embodiment is on the order of about 0.4 microns or less. Other spacings may be appropriate, depending for instance upon the application in which the MEMS flow module 40 is being used. In one embodiment, the height of the gap 58 in the FIG. 2C configuration is no more than about 0.3 microns, although a height of about 0.1 microns or less may be desirable in at least certain instances. These same spacings may be realized/utilized when the annular flow-restricting rings 54 instead extend from the baffles 66 in the above-noted manner. Moreover, the same spacings may be realized/utilized when a particular flow-restricting ring 54 is replaced by a plurality of flow-restricting segments that are appropriately spaced from each other.

The annular flow-restricting rings 54 may be formed in conjunction with the anchors 70 and annular connectors 72. Specifically, annular troughs may be formed through the S₃ layer 24 to the P₂ layer 22 on top of each of the baffles 66. In order to separate the annular flow-restricting rings 54 from the baffles 66, a very thin layer (e.g., about 0.3 microns or less, and corresponding with desired size of the gap 58) of sacrificial material may be deposited on top of the S₃ layer 24 and at the base of these annular troughs. The thickness of this layer is definable at small dimensions. As will be appreciated, formation of the annular troughs corresponding to the annular flow-restricting rings 54 and deposition of the thin layer of sacrificial material may be performed prior to formation of the holes and annular troughs corresponding to the anchors 70 and annular connectors 72. The deposition of the thin layer of sacrificial material results, after the release, in a narrow gap 58 between the top of the baffle 66 and the bottom of the annular flow-restricting ring 54. The thickness of the deposition may be controlled such that the resulting gap 58 (between the bottom surface of the annular flow-restricting ring 54 and the top surface of the baffle 66) substantially restricts flow across the MEMS flow module 40 in the absence of the baffle 66 being deflected from the home position and away from the flow plate 50. Each gap 58 may also define a filter trap gap of sorts for a flow attempting to proceed between the baffles 66 and the flow plate 50. In one embodiment, each gap 58 may filter a flow through the MEMS flow module 40 when the baffles 66 are in the position illustrated in FIG. 2C, while also providing a desired flow restriction through the MEMS flow module 40. Movement of the baffles 66 away from their corresponding flow port 52 in response to the development of at least a certain differential pressure provides a pressure regulation function in that the MEMS flow module 40 will then accommodate a greater flow. When providing this pressure regulation function, the flow-restricting rings 54 may not be providing any significant filtering function. For at least certain applications, the primary function of the flow-restricting rings 54 is to limit the flow rate through the MEMS flow module 40, and not provide a filtering function. Again, however, the flow-restricting rings 54 may provide a filtering function as desired/required.

The gap 58 may be designed such that the annular flow-restricting ring 54 and its corresponding baffle 66 are spaced to allow at least a certain flow through the MEMS flow module 40 without requiring any deflection of the baffles 66. That is, the MEMS flow module 40 may be designed to provide a constantly open flow path that allows at least a certain limited flow through the MEMS flow module 40 at all times. Such a constantly open flow path may be beneficial in at least number of respects. One relates to the case where the MEMS flow module 40 is used to relieve intraocular pressure in an eye (e.g., by being incorporated into an eye implant). In this case, the flow plate 50 of the MEMS flow module 40 could be on the “anterior chamber” side (e.g., the flow of aqueous humor out of the anterior chamber of the patient's eye through the MEMS flow module 40 would be through one or more flow ports 52, and then through the spacing between the baffles 66 and the flow plate 50, and then ultimately out of the MEMS flow module 40). Having the open flow path exist at all times (such that it always has a volume greater than zero) is believed to at least generally mimic the flow of aqueous humor out of the anterior chamber of a patient's eye through the eye's canal of Schlemm. However, the MEMS flow module 40 could be designed so that the baffles 66 are actually disposed directly on their corresponding annular flow-restricting ring 54 until at least a certain differential pressure exists (e.g., a differential pressure “set point”, which may in fact be zero as noted), after which the baffles 66 then would move into spaced relation with the corresponding annular flow-restricting ring 54 to open the flow path.

Each baffle 66 is interconnected at its base or fixed end 76 to the support 64 of the regulating structure 62. See FIGS. 2B and 2C. Opposite of the fixed base 76 is a free end 78 of the baffle 66. The free end 78 of the baffle 66 is operative to move along an at least generally arcuate path in response to the baffle 66 experiencing at least a certain differential pressure. More specifically, the baffle 66 flexes in response to at least a certain pressure differential that exists across the MEMS flow module 40. If the pressure acting on the side of a particular baffle 66 that faces its corresponding flow port 52 is greater than the pressure acting on the opposite side of this baffle 66 by at least a certain amount, this pressure differential will result in a force that is applied to the baffle 66 that is operative to flex the baffle 66 downward in the view shown in FIG. 2C. That is, the baffle 66 flexes away from its corresponding flow port 52 and annular flow-restricting ring 54 to further open a flow path segment within the MEMS flow module 40. This flexing also stores forces or creates stresses in each baffle 66 that may be used to return the same either back toward or to the position illustrated in FIG. 2C as the magnitude of the pressure differential is reduced. That is, the baffles 66 preferably elastically deform as the pressure differential increases above a certain amount, and the elasticity of the baffles 66 may provide a restoring force that at least contributes to the movement of the baffles 66 back toward or to their respective home position (e.g., FIG. 2C), depending upon the magnitude of the reduction of the pressure differential.

The volume of a flow path segment is at least partially dependent upon the flexure of the baffle 66. The further the baffle 66 is flexed away from its corresponding flow port 52, the greater the volume of the flow path segment will be (e.g., up to a certain maximum). Importantly, the movement of the baffle 66 allows the flow rate through the flow port 52 to increase greater than proportionally to an increase in the pressure differential across the MEMS flow module 40. The maximum distance that the baffle 66 is allowed to move away from the flow plate 50 may be controlled, such as by using an appropriate travel limiter or the like (e.g., a mechanical “catch”).

Typically the MEMS flow module 40 will be used in an application where a high pressure source P_{H} (e.g., the anterior chamber of a patient's eye) acts on the top of the flow plate 50 or that surface of the flow plate 50 which projects or faces away from the regulating structure 62, while a typically lower pressure source P_{L} (e.g., the environment) acts on the bottom of the flow plate 50 or that surface of the flow plate 50 which projects toward or faces the regulating structure 62. A change in the pressure from the high pressure source P_{H} may cause one or more of the baffles 66 to move further away from the flow plate 50, which thereby increases the flow rate through the MEMS flow module 40. Preferably, a very small change in the pressure from the high-pressure source P_{H} will allow for greater than a linear change in the flow rate out of the MEMS flow module 40 through the flow ports 52 and past the baffles 66. For instance, a small increase in the pressure of the high pressure source P_{H} may increase the deflection of the baffles 66 (i.e., such that they move further away from the annular flow-restricting rings 54) to provide more than a linear increase in the flow rate through the MEMS flow module 40. That is, there is preferably a non-linear relationship between the flow rate passing through the MEMS flow module 40 and a change in the differential pressure being experienced by the MEMS flow module 40. The flow rate through the flow path segment defined by the space between the baffles 66 and the annular flow-restricting rings 54 should be a function of the cube of the height of this flow path segment, or the gap 58 between the baffles 66 and their corresponding annular flow-restricting ring 54 (at least in the case of laminar flow, which is typically encountered at these dimensions and flow rates). Stated another way, the development of at least a certain change in the differential pressure across a particular baffle 66 will provide greater than a linear increase in the volume of the flow channel segment between the flow-restricting ring 54 and its corresponding baffle 66.

Consider the case where the MEMS flow module 40 is used in an implant to regulate the pressure in the anterior chamber of a patient's eye that is diseased, and where it is desired to maintain the pressure within the anterior chamber of this eye at about 5 mm of HG. The stiffness of the baffles 66 may be configured such that they will adjust the flow rate out of the anterior chamber and through the MEMS flow module 40 such that the maximum pressure within the anterior chamber of the patient's eye should be no more than about 7-8 mm of HG (throughout the range for which the MEMS flow module 40 is designed). Stated another way, the stiffness of the baffles 66 allows for maintaining at least a substantially constant pressure in the anterior chamber of the patient's eye (the high pressure source P_{H} in this instance), at least for a reasonably anticipated range of pressures within the anterior chamber of the patient's eye.

In order to regulate the pressure differential across and/or flow through the MEMS flow module 40, one or more characteristics of the flow ports 52 and/or baffles 66 may be adjusted. As will be appreciated, the force applied to each baffle 66 by a differential pressure is proportional to the area of the corresponding flow port 52. Accordingly, by adjusting the size (e.g., diameter) of the flow ports 52, the force applied to the baffles 66 for a given pressure differential may be increased and/or decreased. Likewise, the stiffness of the baffles 66 may be designed for a particular application. In this regard, the baffles 66 can be likened to a beam having a fixed base 76 and a free end 78. By adjusting the width, height, cross-sectional shape and/or length of such a beam, the stiffness the baffle 66 may be adjusted. The stiffness of the baffles 66 will of course have an effect on the magnitude of the differential pressure that must exist to start flexing the baffles 66.

There are a number of features and/or relationships that contribute to the pressure or flow regulation function of the MEMS flow module 40, and that warrant a summarization. First is that the MEMS flow module 40 is an autonomous or self-contained device. No external power is required for operation of the MEMS flow module 40. Stated another way, the MEMS flow module 40 is a passive device—no external electrical signal of any type need be used to move the baffles 66 relative to the flow plate 50 for the MEMS flow module 40 to provide its pressure or flow regulation function. Instead, the position of the baffles 66 relative to the flow plate 50 is dependent upon the differential pressure being experienced by the baffles 66, and the flow rate out of the MEMS flow module 40 (through the space between adjacent baffles 66 and/or the space between the baffles 66 and the outer support ring 68) is in turn dependent upon the position of the baffles 66 relative to the flow plate 50 (the spacing therebetween (e.g., gap 58), and thereby the size of this flow path segment). Finally, it should be noted that the MEMS flow module 40 may be designed for a laminar flow therethrough, although the MEMS flow module 40 may also be applicable for a turbulent flow therethrough as well.

As will be appreciated, prior to the release of the MEMS flow module 40, at least one sacrificial layer (e.g., the S₃ layer 24) will be disposed between the flow plate 50 and the regulating structure 62, while at least one sacrificial layer (e.g., the S₁ layer 16) will be disposed on the side of the regulating structure 62 that is opposite that which faces the flow plate 50. In order to remove these sacrificial layers, a plurality of etch release holes may be formed through the flow plate 50 and through the regulating structure 62 in order to reduce the amount of time required to remove these sacrificial layers. Typically these etch release holes will have a diameter of no more than about one micron. At least certain lithographic techniques only permit the formation of an etch release hole having a diameter on the order of about one micron. As will be appreciated, such etch release holes will remain in the resulting MEMS flow module 40. There are a number of potential disadvantages associated with etch release holes of this size for the MEMS flow module 40. One is that the existence of a number of etch release holes of this size may provide an undesirably high minimum flow rate through the MEMS flow module 40. That is, etch release holes of this size could possibly have an undesired effect on the flow or pressure regulating capabilities of the MEMS flow module 40. Another is that potentially undesirable contaminants having a size of about one micron or less may pass through the MEMS flow module 40 by passing through such etch release holes.

In cases where the diameter of the etch release holes cannot be made sufficiently small (e.g., a diameter of no more than about 0.2 or 0.3 microns), and possibly depending upon the location of a particular etch release hole in the MEMS flow module 40, a flow-restricting structure or a flow restrictor may be provided in relation to one or more of these etch release holes. A single flow restrictor may be associated with a single etch release hole in a given fabrication level, or may be associated with multiple etch release holes in a given fabrication level. In the case of the MEMS flow module 40, a flow restrictor may be provided for each etch release hole through the flow plate 50. However, a flow restrictor may only be required for those etch release holes through the baffles 66 that are aligned with or encompassed by a corresponding flow port 52 in the flow plate 50. A flow restrictor could be provided for each etch release hole utilized by the MEMS flow module 40, or for any number of etch release holes utilized by the MEMS flow module 40. For instance, a flow restrictor may be used for a certain percentage of the etch release holes through the flow plate 50, and again possibly only for those etch release holes through the baffles 66 that are aligned with or encompassed by a corresponding flow port 52 in the flow plate 50. However, a flow restrictor could be used in relation to any number of etch release holes through a particular baffle 66.

The desire to provide a restricted flow through the MEMS flow module 40 with the baffles 66 being in their home position may be especially important in biological applications, such as where the MEMS flow module 40 isolates a biological reservoir (e.g., an interior chamber of a human eye; a cranial reservoir chamber) from another biological reservoir, the environment, and/or a man-made reservoir. In order to provide a desirable restricted flow through the MEMS flow module 40, appropriate flow restrictors may be formed for any desired etch release hole. FIG. 2D illustrates one embodiment of a flow restrictor 80 that may be formed for an etch release hole 56 through the flow plate 50 and that is located on the side of the flow plate 50 that is opposite the regulating structure 62. It should be appreciated that this same flow restrictor 80, or at least one that is principally the same, may be used elsewhere within the MEMS flow module 40. This flow restrictor 80 is operative to provide a restricted flow through a gap of about 0.4 microns or less. The size of this gap, and thereby the magnitude of the flow restriction, may be selected as desired/required for a particular application. A gap on the order of about 0.3 microns or less may be preferable for at least certain applications. In another embodiment, the gap is on the order of about 0.1 microns or less.

Each such flow restrictor 80 includes a top plate 82 (e.g., formed in the P₄ layer 30), an etch release hole 84 passing through the top plate 82, an annular retaining wall 86 interconnecting the top plate 82 to the flow plate 50, and one or more flow-restricting walls 88 interconnected to the top plate 82 and extending downward towards, but not to the flow plate 50. A single flow-restricting wall 88 could be provided and in the form of an annular structure that extends 360 degrees about a reference axis to define an “interiorly located” closed perimeter for the flow restrictor 80 (the illustrated embodiment). Multiple flow-restricting walls 88 that are appropriately spaced from each other could be utilized as well. The annular retaining wall 86 contains all flow between the etch release hole 84 in the top plate 82 and the etch release hole 56 in the flow plate 50. Accordingly, the etch release hole 56 through the flow plate 50 is disposed within the closed perimeter of the annular retaining wall 86. Likewise, the etch release hole 84 within the top plate 82 is also disposed within the closed perimeter of the annular retaining wall 86. As noted above, current lithographic techniques may not permit creation of etch release holes 56, 84 having a sufficiently small size for purposes of the MEMS flow module 40. Accordingly, the flow restrictor 80 utilizes at least one flow-restricting wall 88 that is disposed within or inwardly of the annular retaining wall 86 to provide a desired flow restriction (and to limit the size of particulates/contaminants that may pass through the flow restrictor 80 if desired/required).

As shown, each flow-restricting wall 88 is fixedly interconnected to the bottom surface of the top plate 82. As with the annular retaining wall 86, the flow-restricting wall 88 may be an annular structure that extends 360 degrees about a reference axis to define a closed perimeter. In the embodiment shown, the etch release hole 84 through the top plate 82 is disposed within or radially inward of the closed perimeter of the annular flow-restricting wall 88, while the etch release hole 56 through the flow plate 50 is disposed outside or radially outward from the closed perimeter of the annular flow-restricting wall 88. The reverse of course could be done as well. The flow-restricting wall 88 extends downwardly towards the surface of the flow plate 50, but does not contact that surface. That is, a gap 90 exists between the top of the flow plate 50 and the lower edge of the flow-restricting wall 88. This gap 90 provides the desired flow restriction for the flow restrictor 80.

As with the annular flow-restricting rings 54 discussed above, the size of this gap 90 can be finely controlled for each flow restrictor 80 to provide a desired flow restriction (and also to provide a spacing that may reduce the potential for undesired contaminants passing completely through the flow restrictor 80 if desired/required). Accordingly, the flow restrictor 80 is formed in a manner similar to the annular flow-restricting rings 54 discussed above. In this regard and in one embodiment, once the flow plate 50 is patterned, a sacrificial layer (e.g., S₄ layer 28) may be deposited on the upper surface of the flow plate 50. A plurality of annular troughs may be formed in the sacrificial layer that extend all the way down to the surface of flow plate 50. These annular troughs will form the annular flow-restricting walls 88 for the various flow restrictors 80. A very thin layer of sacrificial material, for example about 0.3 microns or less, may then be deposited at the base of the annular troughs. This thin layer of sacrificial material dictates the spacing between the bottom of the annular flow-restricting wall 88 and the top surface of the flow plate 50 after the release (i.e., defines the gap 90). Once the thin layer of sacrificial material is deposited, a second set of annular troughs may be formed in the sacrificial layer, that again extend all the way down to the surface of the flow plate 50. These additional annular troughs will form the outer retaining walls 86 for the various flow restrictors 80. Accordingly, the fabrication level that defines the top plate 82 (e.g., the P₄ layer 30) may then be deposited on top of the sacrificial layer (e.g., S₄ layer 28) such that the two sets of cylindrical holes defining the annular retaining walls 86 and annular flow-restricting walls 88 are filled and exist in the same fabrication level that forms the top plate 82 of each flow restrictor 80. This fabrication level may then be patterned to define the individual top plates 82 and etch release holes 84 for the flow restrictors 80.

In this arrangement, fluid has to flow through the etch release hole 84 in the top plate 82 within the closed perimeter of the annular flow-restricting wall 88, through the gap 90 between the bottom of the annular flow-restricting wall 88 and the top of the flow plate 50, and then through the etch release hole 56 within the flow plate 50, or vice versa. As will be appreciated, the construction of the flow restrictor 80 may be reversed such that the annular flow-restricting wall 88 is formed on the top surface of the flow plate 50 and the gap 90 exists between the annular flow-restricting wall 88 and the bottom surface of the top plate 82. Likewise, it is a matter of design choice as to which etch release hole 84, 56 is disposed within the closed perimeter of the annular flow-restricting wall 88. What is important is that one of the etch release holes 56, 84 is disposed within the closed perimeter of the annular flow-restricting wall 88, and that the other is disposed between the annular flow-restricting wall 88 and the annular retaining wall 86. That is, all flow through the flow restrictor 80 is preferably forced to pass through a gap 90 of a desired size. In any case, it may be such that the size of the gap 90 may be definable at smaller dimensions than the sizing of the etch release holes 56, 84 to provide a desired flow restriction.

FIG. 2E illustrates another embodiment of a flow restrictor 92. This flow restrictor 92 may be used for other etch release holes utilized by the MEMS flow module 40, but is illustrated in relation to an etch release hole through one of the baffles 66. The flow restrictor 92 is actually integrated into the configuration of a baffle 66 that is fabricated from both the P₂ layer 22 and the P₁ layer 18. However, the basic configuration/principles of the flow restrictor 92 could be implemented in any pair of spaced fabrication levels in the MEMS flow module 40.

The flow restrictor 92 operates much the same way as the flow restrictor 80 of FIG. 2D. The flow restrictor 92 includes an etch release hole 95 that extends through the P₂ layer 22 and interfaces with a discrete pocket 65 that was formed between part of the interfacing portions of the P₂ layer 22 and P₁ layer 18 that collectively define the baffle 66. An etch release hole 67 extends through the P₁ layer 18 and interfaces with the pocket 65 at a location so as to be offset from the etch release hole 95. The height of the pocket 65 may provide a desired flow restriction. In addition, the P₂ layer 22 may also include a stud 98 that extends into, but that is spaced from, the etch release hole 67 that extends through the P₁ layer 18. The space between the stud 98 and the sidewall of the P₁ layer 18 that defines the etch release hole 67 may also provide a desired flow restriction as well.

As with the above noted flow restrictor 80, the flow restrictor 92 may be formed through a series of patterning, deposition, further patterning, and release steps. Specifically and for the illustrated example where the baffle 66 is fabricated from both the P₂ layer 22 and the P₁ layer 18, the S₂ layer 20 may be deposited on the upper surface of the P₁ layer 18 and in only the lower portion of the etch release hole 67 and along the entire vertical extent of the sidewall of the etch release hole 67. The S₂ layer 20 may be patterned to leave an “island” that will define the perimeter of the pocket 65. Once so patterned, the P₂ layer 22 may be deposited onto the S₂ layer 20 and into the portion of the etch release hole 67 that is not occupied by the S₂ layer 20. Thereafter, the etch release hole 95 may be formed within the P₂ layer 22. The MEMS flow module 40 may then be released using the flow restrictor 92.

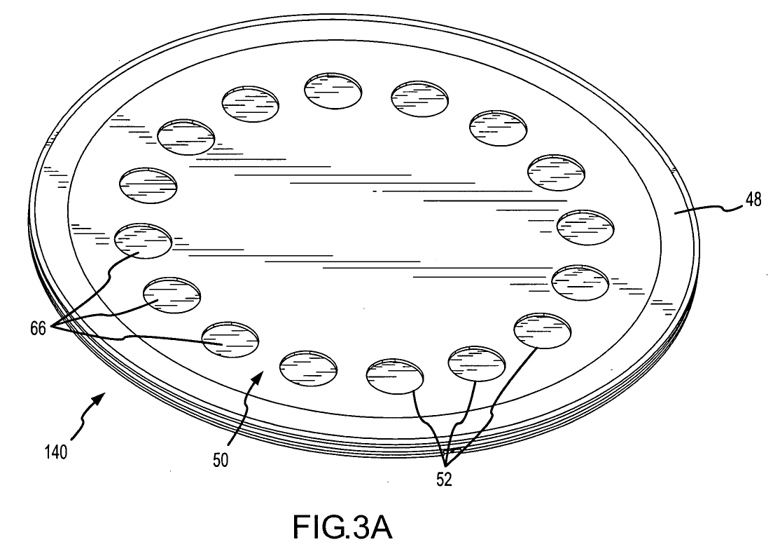

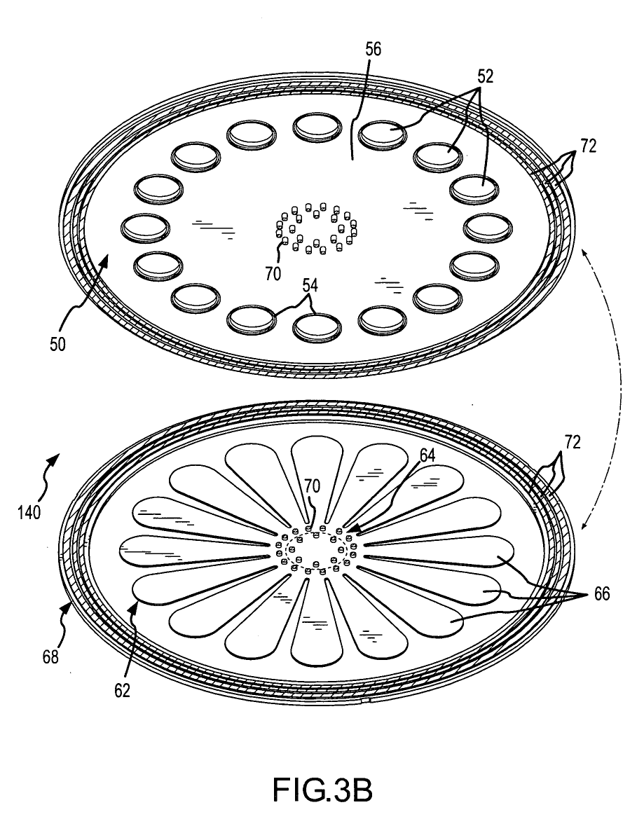

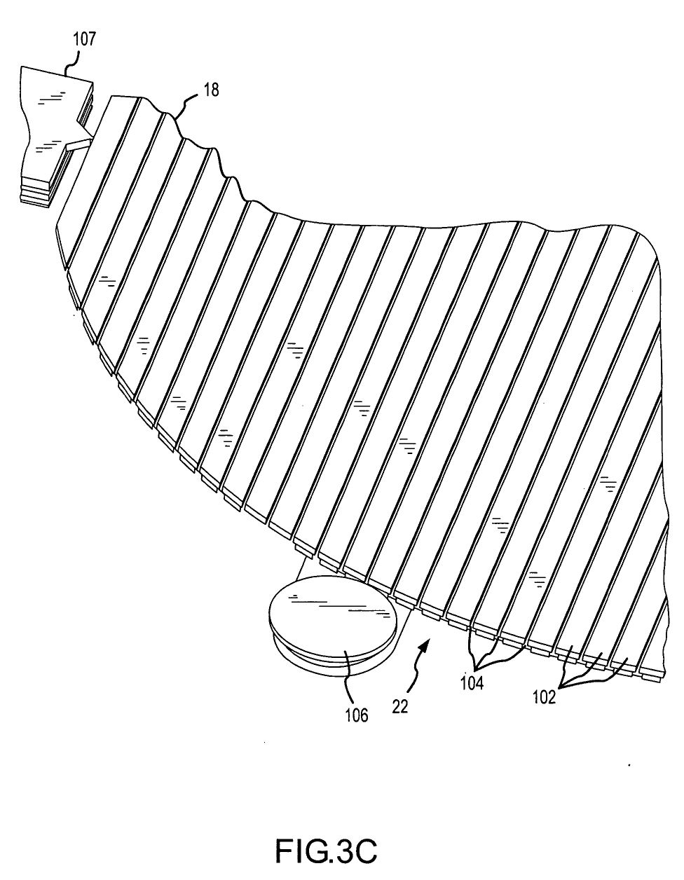

Another embodiment of a MEMS flow module is illustrated in FIGS. 3A-3C and is identified by reference numeral 140. MEMS flow module 140 shares many attributes with the MEMS flow module 40 discussed in relation to FIGS. 2A-2E, and the discussion of corresponding components presented above is applicable to the MEMS flow module 140. The primary difference is that the MEMS flow module 140 is fabricated from different levels than the MEMS flow module 40, and further in a manner that may alleviate the need for etch release holes through the flow plate 50 and baffles 66. Accordingly, like components are labeled with like reference numbers.

The MEMS flow module 140 of FIG. 3A includes a flow plate 50 (e.g., fabricated in the P₄ layer 30) having a plurality of flow ports 52, a regulating structure 62 (e.g., fabricated in the P₃ layer 26), and an outer support ring 68 (e.g., fabricated in the P₃ layer 26). In addition to the MEMS flow module 140 using fabrication levels that are different than those used by the MEMS flow module 40 as described above, the MEMS flow module 140 is fabricated in a manner that may not require any etch release holes through the various baffles 66 and flow plate 50 as noted. In this regard, the flow restrictors as discussed above may not be required for the MEMS flow module 140. However, it will be appreciated that the MEMS flow module 140 is still principally a two fabrication level device, where a layer of sacrificial material is disposed on both sides of the fabrication level that defines both the regulating structure 62 and outer support ring 68, and which must be removed prior to using the MEMS flow module 140 for its intended application.

There will typically be sacrificial material on the side of the regulating structure 62 that is opposite that which faces the flow plate 50 (e.g., the S₃ layer 24), as well as sacrificial material on the side of the regulating structure 62 that faces the flow plate 50 (e.g., the S₄ layer 28). This sacrificial material may be removed in any appropriate manner. FIG. 3C illustrates one way to remove this sacrificial material without having etch release holes through any of the baffles 66 and/or the support 64, so long as there is a sufficient gap between adjacent baffles 66. As shown, at least the P₂ layer 22 is patterned to form a plurality of etch release rails 102 that are spaced from each other, across the entire lateral extent of the MEMS flow module 140, and that are in accordance with the disclosure of U.S. Pat. No. 6,756,317, the entire disclosure of which is incorporated by reference in its entirety herein. These etch release rails 102 also may be fabricated in the P₁ 18 layer as well. More specifically, each of these rails 102 is separated by a trough 104. Generally, each trough 104 may be filled with a sacrificial material from the deposition of the S₃ layer 24 over the P₂ layer 22. The density of the S₃ layer 24 that exists along the vertical walls of the etch release rails 102 is reduced and is etched at a greater rate than the remainder of the S₃ layer 24. The etching of these lower density regions in effect defines etch release conduits that extend along the etch release rails 102 at the start of the release and that then allows for the removal of both the S₃ layer 24 and the S₄ layer 28 at a desired rate in accordance with the above-noted 6 U.S. Pat. No. 6,756,317. Any of the rapid etch release techniques and corresponding structures disclosed by the above-noted U.S. Pat. No. 6,756,317 may be used in the fabrication of the MEMS flow module 140 and the various other MEMS flow modules disclosed herein. Another option for removing the sacrificial material would be to form one or more etch release holes through the substrate (e.g. substrate 10) on which the MEMS flow module 140 is fabricated. These etch release holes could be formed by what is commonly referred to as a back side etch (e.g., using a deep RIE (reactive ion etching) type of tool).

FIG. 3C also illustrates one embodiment of a flow module suspension tab 107 and one embodiment of a motion limiter 106. A plurality of the flow module suspension tabs 107 and a plurality of motion limiters 106 would typically be provided for the MEMS flow module 140. These same types of flow module suspension tabs 107 and motion limiters 106 may be used in relation to the fabrication of the various other MEMS flow modules described herein. Generally, the MEMS flow module 140 is supported above the substrate 10 after the release by the plurality of flow module suspension tabs 107. Each flow module suspension tab 107 is appropriately anchored to the substrate 10 on which the MEMS flow module 140 is fabricated and may engage, for instance, the outer support ring 68 of the MEMS flow module 140. A small force may be exerted on these flow module suspension tabs 107 to structurally disconnect the MEMS flow module 140 from the substrate 10 (e.g., by breaking the tabs 107). A plurality of the noted motion limiters 106 may be disposed about the periphery of the MEMS flow module 140 to limit lateral movement of the MEMS flow module 140 after being structurally disconnected from the substrate 10 and until thereafter removed using a movement that is at least generally away from the substrate 10.

Another embodiment of a MEMS flow module is illustrated in FIGS. 4A-4C and is identified by reference number 240. The MEMS flow module 240 shares many attributes with the MEMS flow modules discussed above, and the discussion of corresponding components presented above is applicable to the MEMS flow module 240. However, the MEMS flow module 240 incorporates an additional flow or reinforcement plate 110 (e.g., formed in the P₄ layer 30) to increase the overall stiffness of the MEMS flow module 240. FIG. 4B is a cross-sectional view taken along a reference plane that extends between the reinforcement plate 110 and the flow plate 50, and with the reinforcement plate 110 being rotated or pivoted away from the flow plate 50. FIG. 4C is the same type of view presented in FIG. 2B, but for the case of the MEMS flow module 240. That is, FIG. 4C is a cross-sectional view taken between the flow plate 50 and the regulating structure 62 so as to extend through the gap 58 between each flow-restricting ring 54 and its corresponding baffle 66.

The reinforcement plate 110 includes a plurality of flow ports 112 that are preferably aligned with the flow ports 52 through flow plate 50, although such is not required. There may be a one-to-one relation between the flow ports 112 and the flow ports 52, although such is not required. In order to increase the structural rigidity of the MEMS flow module 240, continuous annular connectors 114 are disposed between and interconnect the reinforcement plate 110 and the flow plate 50, as illustrated in FIG. 4B. Each annular connector 114 is disposed about the perimeter of its corresponding flow port 52 and the perimeter of its corresponding flow port 112. Each annular connector 114 could be replaced by a plurality of connector segments (not shown) that would be collectively disposed about the corresponding flow port 52 and appropriately spaced from each other. The reinforcement plate 110 and the flow plate 50 are structurally interconnected at other locations as well. For instance, the outside perimeter of the flow plate 50 and reinforcement plate 110 are interconnected by one or more annular connectors 116 in a similar manner to the connection of the outer support ring 68 to the bottom surface of the flow plate 50 discussed above in relation to the embodiment of FIGS. 2A-B. As will be appreciated, the use of multiple, radially spaced, annular connectors 116 between the flow plate 50 and the reinforcement plate 110 again provides redundant “radial” seals for the perimeter of the MEMS flow module 240.

As shown in FIGS. 4A and 4B, the reinforcement plate 110 is also interconnected to the surface of the flow plate 50 by a plurality of anchors 118 that further stiffen the resulting MEMS flow module 240. These anchors 118 extend continuously between the plates 50 and 110, and may be in accordance with the anchors 70 discussed above in relation to FIG. 2B. FIG. 4C shows the interconnection of the flow plate 50 to the regulating structure 62 and outer support ring 68. As shown, the interconnection of these structures is substantially identical to that described above in relation to FIG. 2B.

Another reinforcement option for the MEMS flow module 240 would be to dispose the reinforcement plate 110 in interfacing relation with the flow plate 50. For instance, the flow plate 50 could be fabricated in the P₃ layer 26. The S₄ layer 28 could then be deposited on the flow plate 50 and into the flow ports 52. The portion of the S₄ layer 28 on the upper surface of the flow plate 50 could then be removed. The P₄ layer 30 would then be deposited directly on the upper surface of the flow plate 50. The flow ports 112 could then be patterned so as to intersect with the corresponding flow port 52 or so as to otherwise fluidly interconnect with one or more flow ports 52. This same reinforcement technique could be utilized in relation to other MEMS flow modules described herein.

In order to remove the sacrificial material (e.g., the S₄ layer 28) between the flow plate 50 and the reinforcement plate 110 in the illustrated embodiment and/or the sacrificial material (e.g., the S₃ layer 24) between the flow plate 50 and the regulating structure 62, a plurality of etch release holes may extend through each of the flow plate 50, the regulating structure 62, and/or the reinforcing plate 110. Any appropriate flow restrictor may be used for one or more of these etch release holes and in accordance with the embodiment discussed above in relation to FIGS. 2A-F. Other techniques for facilitating the release may be used in relation to the MEMS flow module 240 as well.

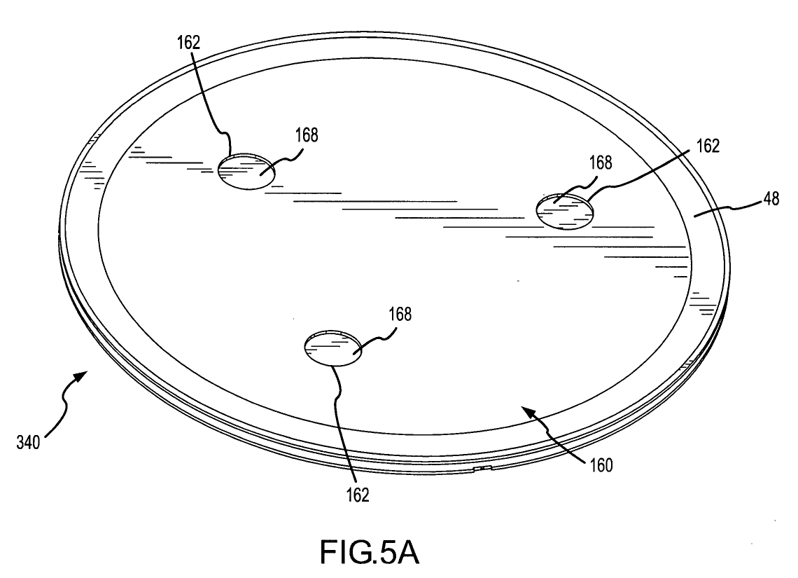

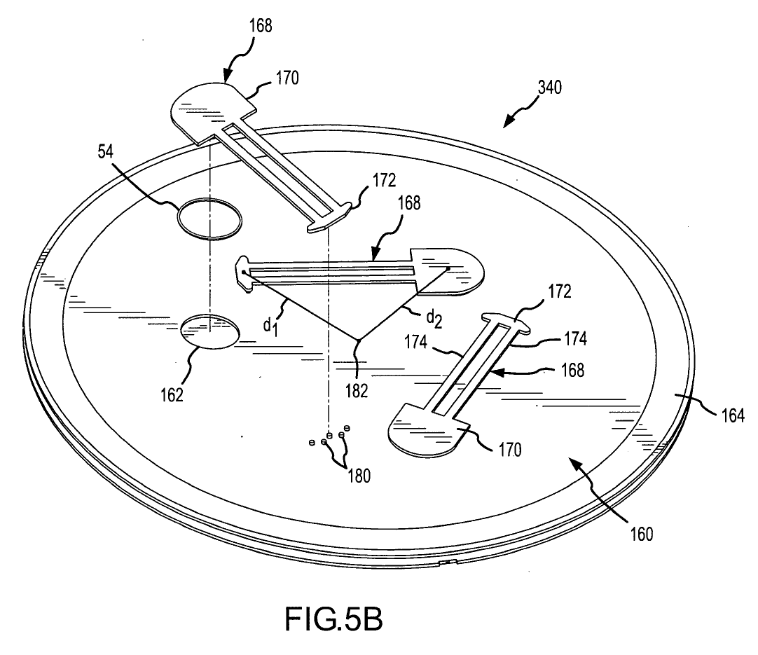

Another embodiment of a MEMS flow module is illustrated in FIGS. 5A and 5B and is identified by reference numeral 340. As shown in FIG. 5A, the MEMS flow module 340 utilizes a flow plate 160 (e.g., fabricated in P₄ layer 30) having three flow ports 162. Any number of flow ports 162 may be utilized, and the flow ports 162 may be otherwise in accordance with the flow ports 52 discussed above in relation to the embodiment of FIGS. 2A-2C. Interconnected to the bottom surface of the flow plate 160 in a manner similar to that discussed above is an outer support ring 164 (e.g., fabricated in P₃ layer 26) and three cantilevered baffles 168 (e.g., fabricated in P₃ layer 26). Any number of cantilevered baffles 168 may be used.

The cantilevered baffles 168 each include a free end portion 170 that is sized to restrict flow through a corresponding flow port 162, and a fixed end portion 172 that is fixedly interconnected to the bottom surface of the flow plate 160 utilizing one or more studs 180 of any appropriate size/shape/configuration/arrangement. That is, instead of having the plurality of baffles 168 be interconnected with a common structure (e.g., a support 64), that in turn is maintained in a fixed positional relationship relative to a flow plate, the baffles 168 are more directly anchored to the flow plate 160. The general orientation of the baffles 168 in the illustrated embodiment may be beneficial in addressing flexing of the flow plate 160 in at least some respect.

Each flow port 162 may have an associated annular flow-restricting ring 54 to reduce the size of a flow path between each of the cantilevered baffles 168 and the flow plate 160. Once again, if the flow plate 160 is fabricated at a level that is further from the substrate 10 than the baffles 168, the annular flow-restricting ring 54 could be attached to and extend from the flow plate 160 (and terminate prior to reaching the corresponding baffle 168). If the flow plate 160 is fabricated at a level that is closer to the substrate 10 than the baffles 168, the annular flow-restricting wall 54 could be attached to and extend from a corresponding baffle 168 (and terminate prior to reaching the flow plate 160).

Interconnecting the free end portion 170 and the fixed end portion 172 of each baffle 168 are two parallel beams or compliant members 174. Although shown as utilizing two parallel beams 174, it will be appreciated that any number and arrangement of one or more beams or compliant members may be utilized. In any case, each baffle 168 flexes such that its free end portion 170 moves along an at least generally arcuate path relative to the flow plate 160 (e.g., at least generally pivots about an axis that is perpendicular to a length dimension of the baffle 168).

The flow module 340 is designed in a manner that addresses manufacturing tolerances and flexure/deflection of the flow plate 160 in response to the existence of at least a certain pressure differential across the MEMS flow module 340. If a cantilevered baffle was fixedly interconnected to the center of a substantially round flow plate, deflection of that flow plate from a static position in response to an applied pressure would generally be near zero at the periphery of the flow plate and increase to a maximum at the center of the flow plate. Accordingly, the unequal deflection across the diameter of the flow plate may make it difficult for such a cantilever-type baffle to function in a manner that provides a desired flow regulation function. The embodiment of FIGS. 5A-B addresses flexing of the flow plate 160 by orienting each baffle 168 such that its fixed end portion 172 and free end portion 170 are disposed at least generally the same distances d₁ and d₂, respectively, from a geometric center 182 of a round flow plate 160. The center of the free end portion 170 of the baffle 168 coincides with the center of its associated flow port 162. In this regard, if the flow plate 160 deflects in response to the development of at least a certain differential pressure, the deflection of the fixed end portion 172 and free end portion 170 will be substantially similar as they are located at least substantially equal distances d₁ and d₂, respectively, from the geometric center 182. Therefore, the free end portion 170 of a particular baffle 168 and the aligned portion of the flow plate 160, namely its associated flow port 162, should move about the same amount due to a deflection of the flow plate 160. As such, the spacing between the free end portion 170 and its corresponding flow port 162 may stay at least generally constant during a flexing of the flow plate 160, assuming that the baffle 168 does not itself flex. Stated otherwise, unequal movement between the free end portion 170 and the fixed end portion 172 of a cantilevered baffle 168 caused by deflection of the flow plate 160 may be substantially reduced or eliminated. That is, as the flow plate 160 flexes, the free end portion 170 and fixed end portion 172 of the cantilevered baffle 168 are displaced an approximately equal distance.

In addition to the foregoing, the baffles 168 do not extend from a common location in the case of the MEMS flow module 340. Although the baffles 168 are still symmetrically disposed about the geometric center 182, the axis along which the length dimension of each such baffle 168 extends does not intersect with this geometric center 182. Instead, the plurality of baffles 168 are arranged so as to collectively define a perimeter or region, and the geometric center 182 is disposed within this region.

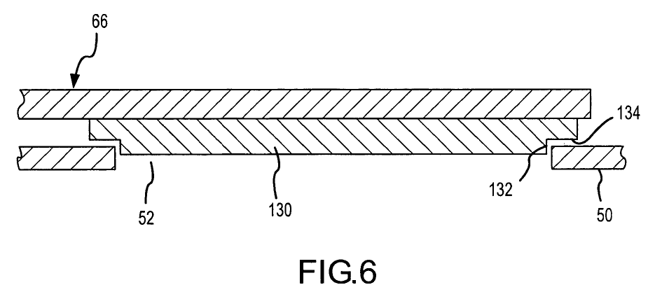

FIG. 6 illustrates a plug 130 that may be utilized with any of the baffles discussed herein above. In this regard, the plug 130 may be formed on the free end of a baffle (e.g., baffle 66) and be sized for disposition within a corresponding flow port (e.g., flow port 52). While sized for disposition within the flow port 52, the plug 130 is preferably generally of a diameter slightly less than the diameter of the flow port 52. Further, the plug 130 may include a stepped edge. In this regard, the plug 130 may include a first portion 132 sized for disposition within the flow port 52 and a second portion 134 that is sized for disposition over the perimeter of the flow port 52 or possibly to contact the flow plate 50. The gap between the outer perimeter of the plug 130 and the sidewall that defines the flow port 52 may provide the desired flow restriction. Any appropriate configuration for the plug 130 that provides such a flow restriction may be utilized (e.g., a “hollow” plug).

Each of the various MEMS flow modules 40 (FIGS. 2A-C), 140 (FIGS. 3A-C), 240 (FIGS. 4A-C), and 340 (FIGS. 5A-B) provide a flow or pressure regulation function by using at least one baffle that moves at least generally along an arcuate path either away from a flow plate (to increase the flow rate through the MEMS flow module in response to at least a certain increase in the differential pressure) or toward the flow plate (to decrease the flow rate through the MEMS flow module in response to at least certain reduction in the differential pressure across the MEMS flow module). Another characterization of this motion is that the baffle undergoes a pivotal or pivotal-like motion. Any manner of achieving this type of movement for a flow-controlling baffle may be utilized. For instance, instead of using a flow-controlling baffle (e.g., baffle 66) that itself flexes to provide the above-noted type of movement, the flow-controlling baffle could be a more rigid structure that is movably interconnected with an appropriate structure to allow the flow-controlling baffle to move along an at least generally arcuate path relative to the flow plate. Such a flow-controlling baffle could interconnect with a beam or member that torsionally deflects to allow the flow-controlling baffle to move in the noted manner. Various types of hinge structures/configurations could also be utilized. However, the above-noted flexing configurations for the flow-controlling baffles may provide one or more advantages.

Surface micromachining is the preferred technology for fabricating the above-described MEMS flow modules having at least one baffle that flexes in response to a flow through a corresponding flow port(s) in a flow plate. In this regard, these MEMS flow module may be fabricated in at least two different levels that are spaced from each other (hereafter a first fabrication level and a second fabrication level). Generally, each of these MEMS flow modules includes a plate with at least one flow port extending therethrough, and each flow port has a baffle associated therewith that moves relative to the plate. Each such baffle may be fabricated at least in the first fabrication level, while the plate may be fabricated in at least the second fabrication level. It should be appreciated that the characterization of the baffle being in a “first fabrication level” and the plate being in the “second fabrication level” by no means requires that the first fabrication level be that which is deposited “first”, and that the second fabrication level be that which is deposited “second.” Moreover, it does not require that the first fabrication level and the second fabrication level be immediately adjacent.

One or both of the baffle and the flow plate each may exist in a single fabrication level or may exist in multiple fabrication levels. “Fabrication level” corresponds with what may be formed by a deposition of a structural material before having to form any overlying layer of a sacrificial material (e.g., from a single deposition of a structural layer or film). In the above-noted first instance, a deposition of a structural material in a single fabrication level may define an at least generally planar layer. Another option regarding the first instance would be for the deposition of a structural material in a single fabrication level to define an at least generally planar portion, plus one or more structures that extend down toward, but not to, the underlying structural layer at the underlying fabrication level. In either situation and prior to an etch release, in at least some cases there will be at least some thickness of sacrificial material disposed between the entirety of the baffle and the plate.

In the above-noted second instance, two or more structural layers or films from adjacent 10 fabrication levels could be disposed in direct interfacing relation (e.g., one directly on the other). Over the region that is to define the first baffle or first plate, this would require removal of the sacrificial material that is deposited on the structural material at one fabrication level before depositing the structural material at the next fabrication level. Another option regarding the above-noted second instance would be to maintain the separation between structural layers or films in different fabrication levels for the first baffle and/or first plate, but provide an appropriate structural interconnection therebetween (e.g., a plurality of columns, posts, or the like extending between adjacent structural layers or films in different, spaced fabrication levels).

With further regard to fabricating the MEMS flow modules at least in part by surface micromachining, each component thereof (including without limitation any flow plate, regulating structure or baffle, reinforcement plate, outer support, etc.) may be fabricated in a structural layer or film at a single fabrication level (e.g., in P₁ layer 18; in P₂ layer 22; in P₃ layer 26; in P₄ layer 30 (FIG. 1 discussed above)). Consider the case of the flow plate 50 in the FIG. 2B embodiment. The annular rings 54 could be fabricated by forming the regulating structure 62 in the P₂ layer 22, depositing the S₃ layer 24, forming annular troughs in the S₃ layer 24 that extend all the way down to the P₂ layer 22, depositing sacrificial material in the bottom of these annular troughs (the thickness of which will define the spacing between the annular rings 54 and the baffles 66 of the regulating structure 62 illustrated in FIG. 2B), and then depositing the P₃ layer 26 on top of the S₃ layer 24, as well as into the “partially filled” annular troughs in the S₃ layer 24. The deposition of structural material into these “partially filled” annular troughs in the S₃ layer 24 is then what defines the annular rings 54. The flow plate 50 may then be characterized as existing in a single fabrication level (P₃ layer 26 in the noted example), since it was defined by a deposition of a structural material before having to form any overlying layer of a sacrificial material (e.g., from a single deposition of a structural layer or film). It should be noted that at least part of the S₃ layer 24 remains between the entirety of the regulating structure 62 and the flow plate 50 (prior to the etch release).

Each such component of the MEMS flow modules described herein could also be fabricated in multiple structural layers or films at multiple fabrication levels as noted. For instance, the flow plate of a given MEMS flow module could be fabricated in both the P₂ layer 22 and P₁ layer 18, where the P₂ layer 22 is deposited directly on the P₁ layer 18. Another option would be to form a particular component of a given MEMS flow module in multiple structural layers or films at different fabrication levels, but that are structurally interconnected in an appropriate manner (e.g., by one or more posts, columns or the like extending between). For instance, the reinforcing plate 110 or flow plate 50, could be formed in both the P₄ layer 30 and the P₃ layer 26 discussed above in relation to, for example, FIG. 4B, with one or more structural interconnections extending therebetween (that would pass through the S₄ layer 28). Generally, this can be done by forming appropriate cuts or openings down through the S₄ layer 28 (to expose the underlying P₃ layer 26 and that will define such structural interconnections once the P₄ layer 30 is deposited therein) before depositing the P₄ layer 30.

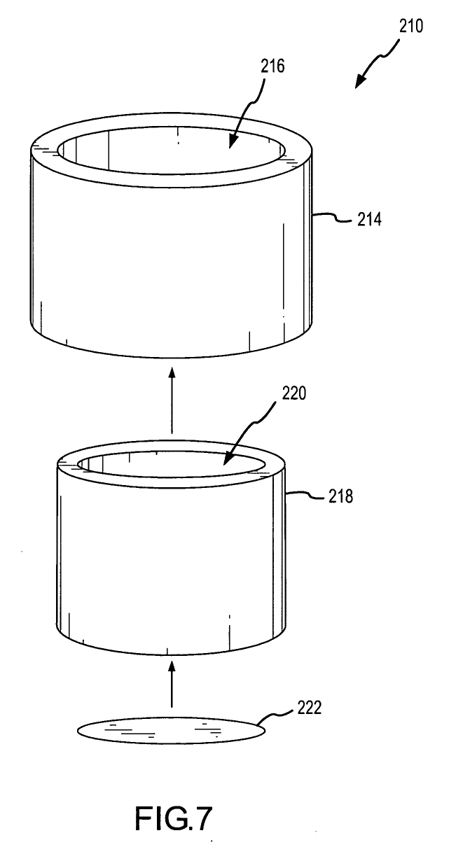

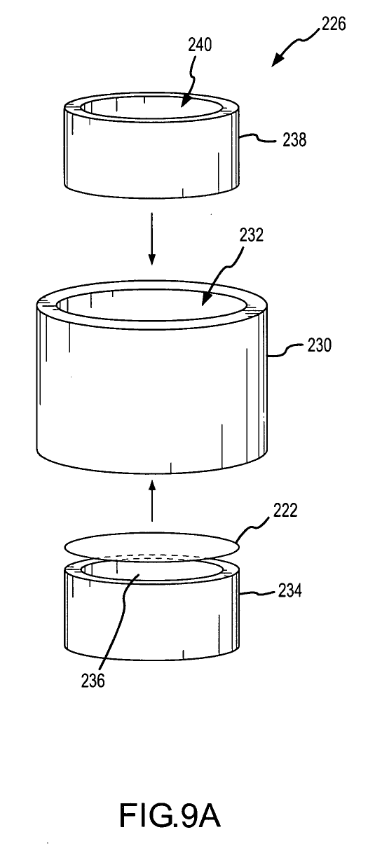

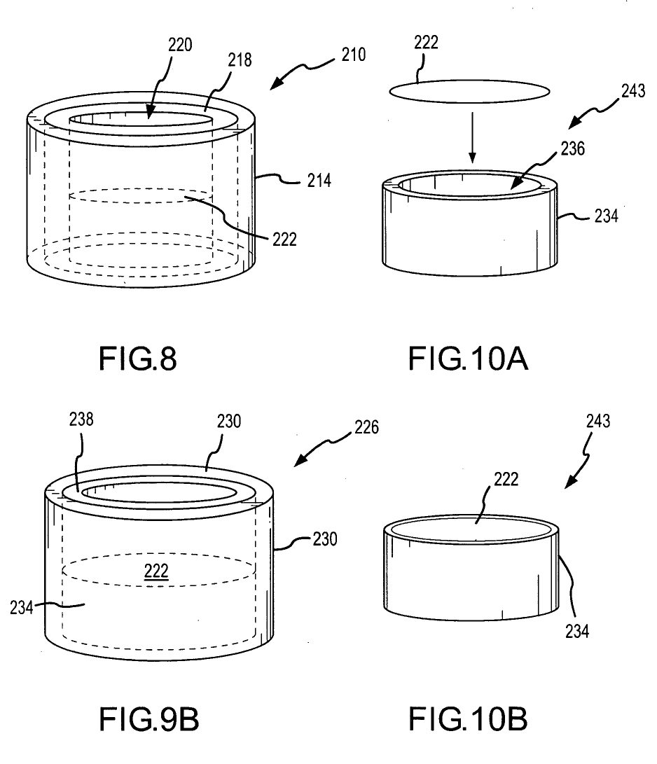

FIGS. 7-8 schematically represent one embodiment of a flow assembly 210 that may be used for any appropriate application (e.g., the flow assembly 210 may be disposed in a flow of any type, may be used to filter and/or control the flow of a fluid of any type, may be located in a conduit that fluidly interconnects multiple sources of any appropriate type (e.g., between multiple fluid or pressure sources (including where one is the environment), such as a man-made reservoir, a biological reservoir, the environment, or any other appropriate source, or any combination thereof). One example would be to dispose the flow assembly 210 in a conduit extending between the anterior chamber of an eye and a location that is exterior of the cornea of the eye. Another example would be to dispose the flow assembly 210 in a conduit extending between the anterior chamber of an eye and another location that is exterior of the sclera of the eye. Yet another example would be to dispose the flow assembly 210 in a conduit extending between the anterior chamber of an eye and another location within the eye (e.g., into Schlemm's canal) or body.

Components of the flow assembly 210 include an outer housing 214, an inner housing 218, and a MEMS flow module 222. Any of the MEMS flow modules described herein may be used in place of the MEMS flow module 222, including without limitation MEMS flow modules 40, 140, 240, or 340. The position of the MEMS flow module 222 and the inner housing 218 are at least generally depicted within the outer housing 214 in FIG. 8 to show the relative positioning of these components in the assembled condition—not to convey that the outer housing 214 needs to be in the form of a transparent structure. All details of the MEMS flow module 222 and the inner housing 218 are not necessarily illustrated in FIG. 8.

The MEMS flow module 222 is only schematically represented in FIGS. 7-8, and provides at least one of a filtering function and a pressure or flow regulation function. The MEMS flow module 222 may be of any appropriate design, size, shape, and configuration, and further may be formed from any material or combination of materials that are appropriate for use by the relevant microfabrication technology. Any appropriate coating or combination of coatings may be applied to exposed surfaces of the MEMS flow module 222 as well. For instance, a coating may be applied to improve the biocompatibility of the MEMS flow module 222, to make the exposed surfaces of the MEMS flow module 222 more hydrophilic, to reduce the potential for the MEMS flow module 222 causing any bio-fouling, or any combination thereof. In one embodiment, a self assembled monolayer coating (e.g., poly-ethylene-glycol) is applied in any appropriate manner (e.g., liquid or vapor phase, with vapor phase being the preferred technique) to all exposed surfaces of the MEMS flow module 222. The main requirement of the MEMS flow module 222 is that it is a MEMS device.

The primary function of the outer housing 214 and inner housing 218 is to provide structural integrity for the MEMS flow module 222 or to support the MEMS flow module 222, and further to protect the MEMS flow module 222. In this regard, the outer housing 214 and inner housing 218 each will typically be in the form of a structure that is sufficiently rigid to protect the MEMS flow module 222 from being damaged by the forces that reasonably could be expected to be exerted on the flow assembly 210 during its assembly, as well as during use of the flow assembly 210 in the application for which it was designed.

The inner housing 218 includes a hollow interior or a flow path 220 that extends through the inner housing 218 (between its opposite ends in the illustrated embodiment). The MEMS flow module 222 may be disposed within the flow path 220 through the inner housing 218 in any appropriate manner and at any appropriate location within the inner housing 218 (e.g., at any location so that the inner housing 218 is disposed about the MEMS flow module 222). Preferably, the MEMS flow module 222 is maintained in a fixed position relative to the inner housing 218. For instance, the MEMS flow module 222 may be attached or bonded to an inner sidewall or a flange formed on this inner sidewall of the inner housing 218, a press-fit could be provided between the inner housing 218 and the MEMS flow module 222, or a combination thereof. The MEMS flow module 222 also could be attached to an end of the inner housing 218 in the manner of the embodiment of FIGS. 10A-B that will be discussed in more detail below.

The inner housing 218 is at least partially disposed within the outer housing 214 (thereby encompassing having the outer housing 214 being disposed about the inner housing 218 along the entire length of the inner housing 218, or only along a portion of the length of the inner housing 218). In this regard, the outer housing 214 includes a hollow interior 216 for receiving the inner housing 218, and possibly to provide other appropriate functionality (e.g., a flow path fluidly connected with the flow path 220 through the inner housing 218). The outer and inner sidewalls of the outer housing 214 may be cylindrical or of any other appropriate shape, as may be the outer and inner sidewalls of the inner housing 218. The inner housing 218 may be retained relative to the outer housing 214 in any appropriate manner. For instance, the inner housing 218 may be attached or bonded to an inner sidewall of the outer housing 214, a press-fit could be provided between the inner housing 218 and the outer housing 214, a shrink fit could be provided between the outer housing 214 and the inner housing 218, or a combination thereof.

The inner housing 218 is likewise only schematically represented in FIGS. 7-8, and it may be of any appropriate shape/configuration, of any appropriate size, and formed from any material or combination of materials (e.g., polymethylmethacrylate (PMMA), titanium, and other implantable metals and plastics). Typically its outer contour will be adapted to match the inner contour of the outer housing 214 in which it is at least partially disposed. In one embodiment, the illustrated cylindrical configuration for the inner housing 218 is achieved by cutting an appropriate length from hypodermic needle stock. The inner housing 218 also may be microfabricated into the desired/required shape (e.g., using at least part of a LIGA process). However, any way of making the inner housing 218 may be utilized. It should also be appreciated that the inner housing 218 may include one or more coatings as desired/required as well (e.g., an electroplated metal; a coating to improve the biocompatibility of the inner housing 218, to make the exposed surfaces of the inner housing 218 more hydrophilic, to reduce the potential for the inner housing 218 causing any bio-fouling, or any combination thereof). In one embodiment, a self assembled monolayer coating (e.g., poly-ethylene-glycol) is applied in any appropriate manner (e.g., liquid or vapor phase, with vapor phase being the preferred technique) to all exposed surfaces of the inner housing 218.

The outer housing 214 likewise is only schematically represented in FIGS. 7-8, and it may be of any appropriate shape/configuration, of any appropriate size, and formed from any material or combination of materials (e.g., polymethylmethacrylate (PMMA), titanium, and other implantable metals and plastics). Typically its outer contour will be adapted to match the inner contour of the housing or conduit in which it is at least partially disposed or otherwise mounted. The outer housing 214 also may be microfabricated into the desired/required shape (e.g., using at least part of a LIGA process). However, any way of making the outer housing 214 may be utilized. It should also be appreciated that the outer housing 214 may include one or more coatings as desired/required as well (e.g., an electroplated metal; a coating to improve the biocompatibility of the outer housing 214, to make the exposed surfaces of the outer housing 214 more hydrophilic, to reduce the potential for the outer housing 214 causing any bio-fouling, or any combination thereof). In one embodiment, a self assembled monolayer coating (e.g., poly-ethylene-glycol) is applied in any appropriate manner (e.g., liquid or vapor phase, with vapor phase being the preferred technique) to all exposed surfaces of the outer housing 214.

Another embodiment of a flow assembly is illustrated in FIGS. 9A-B (only schematic representations), and is identified by reference numeral 226. The flow assembly 226 may be used for any appropriate application (e.g., the flow assembly 226 may be disposed in a flow of any type, may be used to filter and/or control the flow of a fluid of any type, may be located in a conduit that fluidly interconnects multiple sources of any appropriate type (e.g., multiple fluid or pressure sources (including where one is the environment), such as a man-made reservoir, a biological reservoir, the environment, or any other appropriate source, or any combination thereof). The above-noted applications for the flow assembly 210 are equally applicable to the flow assembly to 226. The types of coatings discussed above in relation to the flow assembly 210 may be used by the flow assembly 226 as well.

Components of the flow assembly 226 include an outer housing 230, a first inner housing 234, a second inner housing 238, and the MEMS flow module 222. The MEMS flow 222 and the inner housings 234, 238 are at least generally depicted within the outer housing 230 in FIG. 9B to show the relative positioning of these components in the assembled condition—not to convey that the outer housing 230 needs to be in the form of a transparent structure. All details of the MEMS flow module 222 and the inner housings 234, 238 are not necessarily illustrated in FIG. 9B.

The primary function of the outer housing 230, first inner housing 234, and second inner housing 238 is to provide structural integrity for the MEMS flow module 222 or to support the MEMS flow module 222, and further to protect the MEMS flow module 222. In this regard, the outer housing 230, first inner housing 234, and second inner housing 238 each will typically be in the form of a structure that is sufficiently rigid to protect the MEMS flow module 222 from being damaged by the forces that reasonably could be expected to be exerted on the flow assembly 226 during its assembly, as well as during use of the flow assembly 226 in the application for which it was designed.

The first inner housing 234 includes a hollow interior or a flow path 236 that extends through the first inner housing 234. Similarly, the second inner housing 238 includes a hollow interior or a flow path 240 that extends through the second inner housing 238. The first inner housing 234 and the second inner housing 238 are disposed in end-to-end relation, with the MEMS flow module 222 being disposed between adjacent ends of the first inner housing 234 and the second inner housing 238. As such, a flow progressing through the first flow path 236 to the second flow path 240, or vice versa, passes through the MEMS flow module 222.

Preferably, the MEMS flow module 222 is maintained in a fixed position relative to each inner housing 234, 238, and its perimeter does not protrude beyond the adjacent sidewalls of the inner housings 234, 238 in the assembled and joined condition. For instance, the MEMS flow module 222 may be bonded to at least one of, but more preferably both of, the first inner housing 234 (more specifically one end thereof) and the second inner housing 238 (more specifically one end thereof) to provide structural integrity for the MEMS flow module 222 (e.g., using cyanoacrylic esters, UV-curable epoxies, or other epoxies). Another option would be to fix the position the MEMS flow module 222 in the flow assembly 226 at least primarily by fixing the position of each of the inner housings 234, 238 relative to the outer housing 230 (i.e., the MEMS flow module 222 need not necessarily be bonded to either of the housings 234, 238). In one embodiment, an elastomeric material may be disposed between the MEMS flow module 222 and the first inner housing 234 to allow the first inner housing 234 with the MEMS flow module 222 disposed thereon to be pushed into the outer housing 230 (e.g., the elastomeric material is sufficiently “tacky” to at least temporarily retain the MEMS flow module 222 in position relative to the first inner housing 234 while being installed in the outer housing 230). The second inner housing 238 also may be pushed into the outer housing 230 (before, but more likely after, the first inner housing 234 is disposed in the outer housing 230) to “sandwich” the MEMS flow module 222 between the inner housings 234, 238 at a location that is within the outer housing 230 (i.e., such that the outer housing 230 is disposed about MEMS flow module 222). The MEMS flow module 222 would typically be contacted by both the first inner housing 234 and the second inner housing 238 when disposed within the outer housing 230. Fixing the position of each of the first inner housing 234 and the second inner housing 238 relative to the outer housing 230 will thereby in effect fix the position of the MEMS flow module 222 relative to the outer housing 230. Both the first inner housing 234 and second inner housing 238 are at least partially disposed within the outer housing 230 (thereby encompassing the outer housing 230 being disposed about either or both housings 234, 238 along the entire length thereof, or only along a portion of the length of thereof), again with the MEMS flow module 222 being located between the adjacent ends of the first inner housing 234 and the second inner housing 238. In this regard, the outer housing 230 includes a hollow interior 232 for receiving at least part of the first inner housing 234, at least part of the second inner housing 238, and the MEMS flow module 222 disposed therebetween, and possibly to provide other appropriate functionality (e.g., a flow path fluidly connected with the flow paths 236, 240 through the first and second inner housings 234, 238, respectively). The outer and inner sidewalls of the outer housing 230 may be cylindrical or of any other appropriate shape, as may be the outer and inner sidewalls of the inner housings 234, 238. Both the first inner housing 234 and the second inner housing 238 may be secured to the outer housing 230 in any appropriate manner, including in the manner discussed above in relation to the inner housing 218 and the outer housing 214 of the embodiment of FIGS. 7-8.

Each inner housing 234, 238 is likewise only schematically represented in FIGS. 9A-B, and each may be of any appropriate shape/configuration, of any appropriate size, and formed from any material or combination of materials in the same manner as the inner housing 218 of the embodiment of FIGS. 7-8. Typically the outer contour of both housings 234, 238 will be adapted to match the inner contour of the outer housing 230 in which they are at least partially disposed. In one embodiment, the illustrated cylindrical configuration for the inner housings 234, 238 is achieved by cutting an appropriate length from hypodermic needle stock. The inner housings 234, 238 each also may be microfabricated into the desired/required shape (e.g., using at least part of a LIGA process). However, any way of making the inner housings 234, 238 may be utilized. It should also be appreciated that the inner housings 234, 238 may include one or more coatings as desired/required as well in accordance with the foregoing.

The outer housing 230 is likewise only schematically represented in FIGS. 9A-B, and it may be of any appropriate shape/configuration, of any appropriate size, and formed from any material or combination of materials in the same manner as the outer housing 214 of the embodiment of FIGS. 7-8. Typically the outer contour of the outer housing 230 will be adapted to match the inner contour of the housing or conduit in which it is at least partially disposed or otherwise mounted. The outer housing 230 may be microfabricated into the desired/required shape (e.g., using at least part of a LIGA process). However, any way of making the outer housing 230 may be utilized. It should also be appreciated that the outer housing 230 may include one or more coatings as desired/required in accordance with the foregoing.

Another embodiment of a flow assembly is illustrated in FIGS. 10A-B (only schematic representations), and is identified by reference numeral 243. The flow assembly 243 may be used for any appropriate application (e.g., the flow assembly 243 may be disposed in a flow of any type, may be used to filter and/or control the flow of a fluid of any type, may be located in a conduit that fluidly interconnects multiple sources of any appropriate type (e.g., between multiple fluid or pressure sources, such as a man-made reservoir, a biological reservoir, the environment, or any other appropriate source, or any combination thereof). Components of the flow assembly 243 include the above-noted housing 234 and the MEMS flow module 222 from the embodiment of FIGS. 9A-B. In the case of the flow assembly 243, the MEMS flow module 222 is attached or bonded to one end of the housing 234 (e.g., using cyanoacrylic esters, UV-curable epoxies, or other epoxies). The flow assembly 243 may be disposed within an outer housing in the manner of the embodiments of FIGS. 7-9B, or could be used “as is.” The above-noted applications for the flow assembly 210 are equally applicable to the flow assembly 243. The types of coatings discussed above in relation to the flow assembly 210 may be used by the flow assembly 243 as well.

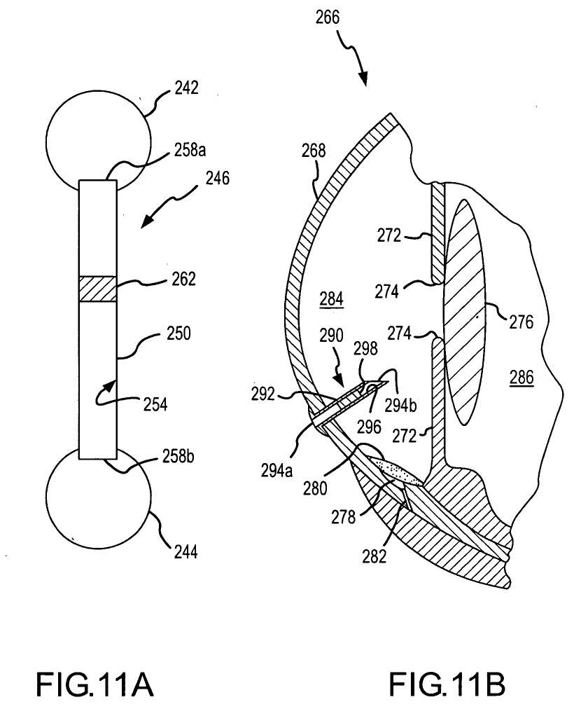

One particularly desirable application for the flow assemblies 210, 226, and 243 of FIGS. 7-10B, as discussed above, is to regulate pressure within the anterior chamber of an eye. This is schematically illustrated in FIG. 11A. Here, an anterior chamber 242 of a patient's eye (or other body region for that matter—a first body region) is fluidly interconnected with an appropriate drainage area 244 by an implant 246. The drainage area 244 may be any appropriate location, such as externally of the eye (e.g., on an exterior surface of the cornea), within the eye (e.g., Schlemm's canal), or within the patient's body in general (a second body region).

Generally, the implant 246 includes a conduit 250 having a pair of ends 258a, 258b, with a flow path 254 extending therebetween. The size, shape, and configuration of the conduit 250 may be adapted as desired/required, including to accommodate the specific drainage area 244 being used. Representative configurations for the conduit 250 are disclosed in U.S. Patent Application Publication No. 2003/0212383, as well as U.S. Pat. Nos. 3,788,327; 5,743,868; 5,807,302; 6,626,858; 6,638,239; 6,533,768; 6,595,945; 6,666,841; and 6,736,791, the entire disclosures of which are incorporated by reference in their entirety herein.

A flow assembly 262 is disposed within the flow path 254 of the conduit 250. All flow leaving the anterior chamber 242 through the implant 246 is thereby directed through the flow assembly 262. Similarly, any flow from the drainage area 244 into the implant 246 will have to pass through the flow assembly 262. The flow assembly 262 may be retained within the conduit 250 in any appropriate manner and at any appropriate location (e.g., it could be disposed on either end 258a, 258b, or any intermediate location therebetween). The flow assembly 262 may be in the form of any of the flow assemblies 210, 226, or 243 discussed above, replacing the MEMS flow module 222 with any of the MEMS flow modules 40, 140, 240, 340 in accordance with FIGS. 1-6. Alternatively, the flow assembly 262 could simply be in the form of the MEMS flow modules 40, 140, 240, or 340. Any appropriate coating may be applied to at least those surfaces of the implant 246 that would be exposed to biological material/fluids, including without limitation a coating that improves biocompatibility, that makes such surfaces more hydrophilic, and/or that reduces the potential for bio-fouling. In one embodiment, a self assembled monolayer coating (e.g., poly-ethylene-glycol) is applied in any appropriate manner (e.g., liquid or vapor phase, with vapor phase being the preferred technique) to the noted surfaces.

FIG. 11B illustrates a representative embodiment in accordance with FIG. 11A. Various portions of the eye 266 are identified in FIG. 11B, including the cornea 268, iris 272, pupil 274, lens 276, anterior chamber 284, posterior chamber 286, Schlemm's canal 278, trabecular meshwork 280, and aqueous veins 282. Here, an implant or shunt 290 having an appropriately-shaped conduit 292 is directed through the cornea 268. The conduit 292 may be in any appropriate form, but will typically include at least a pair of ends 294a, 294b, as well as a flow path 296 extending therebetween. End 294a is disposed on the exterior surface of the cornea 268, while end 294b is disposed within the anterior chamber 284 of the eye 266.

A flow assembly 298 is disposed within the flow path 296 of the conduit 292. All flow leaving the anterior chamber 284 through the shunt 290 is thereby directed through the flow assembly 298. Similarly, any flow from the environment back into the shunt 290 will have to pass through the flow assembly 298 as well. The flow assembly 298 may be retained within the conduit 292 in any appropriate manner and at any appropriate location (e.g., it could be disposed on either end 294a, 294b, or any an intermediate location therebetween). The flow assembly 298 may be in the form of any of the flow assemblies 210, 226, or 243 discussed above, replacing the MEMS flow module 222 with any of the MEMS flow modules 40, 140, 240, 340 in accordance with FIGS. 1-6. Alternatively, the flow assembly 298 could simply be in the form of the MEMS flow modules 40, 140, 240, or 340. Any appropriate coating may be applied to at least those surfaces of the shunt 290 that would be exposed to biological material/fluids, including without limitation a coating that improves biocompatibility, that makes such surfaces more hydrophilic, and/or that reduces the potential for bio-fouling. In one embodiment, a self assembled monolayer coating (e.g., poly-ethylene-glycol) is applied in any appropriate manner (e.g., liquid or vapor phase, with vapor phase being the preferred technique) to the noted surfaces.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

What is claimed is:

1. A MEMS flow module, comprising:
a first plate comprising a first flow port;
a structure comprising a first portion disposed in a fixed positional relationship relative to said first plate and a second portion that at least partially extends over said first flow port, wherein said second portion flexes relative to said first portion in response to the development of at least a certain differential pressure.

2. A MEMS flow module, as claimed in claim 1, further comprising:
at least one anchor extending between said first portion of said structure and said first plate to fixedly interconnect said first portion of said structure with said first plate.

3. A MEMS flow module, as claimed in claim 1, wherein said structure comprises an elongate structure.

4. A MEMS flow module, as claimed in claim 3, wherein said elongate structure further comprises:
a first free end, wherein said second portion is disposed between said first portion and said first free end.

5. A MEMS flow module, as claimed in claim 4, wherein said first free end moves along an at least generally arcuate path in response to the development of at least a certain differential pressure across said second portion of said structure.

6. A MEMS flow module, as claimed in claim 4, wherein said elongate structure further comprises at least one of a cross-sectional shape and a length designed to provide a predetermined resistance to flexure.

7. A MEMS flow module, as claimed in claim 1, wherein said structure and said first plate are formed from adjacent structural MEMS layers.

8. A MEMS flow module, as claimed in claim 1, wherein said second portion of said structure is sized to overly said first flow port when said second portion of said structure is disposed at least generally adjacent to said first flow port.

9. A MEMS flow module, as claimed in claim 8, wherein said second portion of said structure at least substantially blocks a flow through said first flow port in one direction.

10. A MEMS flow module, as claimed in claim 1, wherein said second portion of said structure is substantially parallel to said first plate until the development of at least a certain differential pressure across said second portion.

11. A MEMS flow module, as claimed in claim 1, wherein said second portion of said structure is always disposed in a spaced relationship with said first plate.

12. A MEMS flow module, as claimed in claim 1, wherein a flow-controlling gap of no more than about 0.3 microns exists between said second portion of said structure and said first flow port, until the development of at least a certain differential pressure across said second portion.

13. A MEMS flow module, as claimed in claim 1, wherein the development of at least a certain differential pressure across said second portion of said structure flexes said second portion of said structure away from said first flow port to increase a spacing between said second portion of said structure and said first flow port, and thereby increases a volume of a flow path through said MEMS flow module.

14. A MEMS flow module, as claimed in claim 13, wherein said volume of said flow path increases greater than proportionally for a corresponding increase in a differential pressure across said MEMS flow module.

15. A MEMS flow module, as claimed in claim 1, wherein said first plate comprises a plurality of said first flow ports.

16. A MEMS flow module, as claimed in claim 15, wherein said structure comprises a plurality of said second portions that each at least partially extend over at least one of said plurality of said first flow ports.

17. A MEMS flow module, as claimed in claim 15, further comprising:
a corresponding plurality of said structures, wherein said second portion of each said structure at least partially extends over one of said plurality of said first flow ports, wherein said first portion of each of said plurality of structures are located at substantially at a common first distance from a common point, and wherein said second portion of each of said plurality of structures are located at substantially said first distance from said common point.

18. A MEMS flow module, as claimed in claim 1, further comprising:
a second plate comprising a second flow port, wherein said first and second plates are fixedly interconnected in a spaced and face-to-face relationship.

19. A MEMS flow module, as claimed in claim 18, wherein said first flow port and said second flow port are at least partially aligned.

20. A MEMS flow module, as claimed in claim 1, wherein:
said MEMS flow module is a passive device.

21. A MEMS flow module, as claimed in claim 1, wherein said second portion of said structure further comprises:
a plug structure for disposition within at least a portion of said first flow port.

22. A MEMS flow module, as claimed in claim 1, further comprising:
a flow-restricting structure associated with said first flow port, wherein said flow-restricting structure is disposed somewhere between said first plate and said second portion of said structure.

23. An implant for addressing pressure within a first body region, comprising said MEMS flow module of claim 1 and a conduit, wherein said conduit comprises a flow path that is adapted to fluidly interconnect with the first body region, and wherein said MEMS flow module is disposed in said flow path.

24. A MEMS flow module, comprising:
a first plate comprising a first flow port;
a cantilever structure comprising a first portion that is disposed in a fixed positional relationship relative to said first plate, as well as a free end that is operative to move along an at least generally arcuate path in response to the development of at least a certain differential pressure across said cantilever structure.

25. A MEMS flow module, as claimed in claim 24, wherein a beam portion of said cantilever structure is disposed between said first portion and said first free end.

26. A MEMS flow module, as claimed in claim 24, wherein said beam portion of said cantilever structure is disposed at least generally adjacent to said first flow port until the development of at least a certain differential pressure across said cantilever structure.

27. A MEMS flow module, as claimed in claim 24, wherein a flow rate out of said MEMS flow module increases greater than proportionally for a corresponding increase in a differential pressure across said MEMS flow module.

28. A MEMS flow module, as claimed in claim 24, further comprising;
a plurality of said first flow ports through said first plate.

29. A MEMS flow module, as claimed in claim 28, wherein said cantilever structure comprises a plurality of said free ends, wherein each said free end is associated with at least one said first flow port.

30. A MEMS flow module, as claimed in claim 29, wherein each said free end corresponds with a separate beam portion extending between said fixed portion and each said free end, wherein each said separate beam portion is disposed relative to at least one of said plurality of said first flow ports.

31. A MEMS flow module, as claimed in claim 28, further comprising:
a plurality of said cantilever structures, wherein each said free end is operative to move along an at least generally arcuate path in response to the development of at least a certain differential pressure across said cantilever structure.

32. A MEMS flow module, as claimed in claim 24, wherein said cantilever structure permits a flow through said MEMS flow module in a first direction and substantially restricts a flow through said MEMS flow module in a second direction that is opposite said first direction.

33. A MEMS flow module, as claimed in claim 24, further comprising:
a second plate comprising a second flow port, wherein said first and second plates are fixedly interconnected in a spaced and face-to-face relationship.

34. A MEMS flow module, as claimed in claim 33, wherein said first flow port in said first plate and said second flow port in said second plate are at least partially aligned.

35. An implant for addressing pressure within a first body region, comprising said MEMS flow module of claim 24 and a conduit, wherein said conduit comprises a flow path that is adapted to fluidly interconnect with the first body region, and wherein said MEMS flow module is disposed in said flow path.

36. A MEMS flow module, comprising:
a flow regulator that comprises a plurality of independently movable baffles; and
a first plate that comprises a plurality of first flow ports, wherein each said baffle is aligned with at least one said first flow port, wherein each said baffle is at least generally pivotable to change a magnitude of a spacing of said baffle from said first plate in response to a change in differential pressure across said MEMS flow module.

37. A MEMS flow module, as claimed in claim 36, wherein said flow regulator comprises a support that is spaced from said first plate, wherein said a plurality of baffles are interconnected with said support, and wherein said first plate is structurally interconnected with said support.

38. A MEMS flow module, as claimed in claim 37, wherein said support comprises a perimeter, wherein an entirety of a region disposed inwardly of said perimeter is occupied by said support.

39. A MEMS flow module, as claimed in claim 37, wherein said support comprises an aperture and an annular section disposed about said aperture, wherein said plurality of baffles are interconnected with said annular section.

40. A MEMS flow module, as claimed in claim 37, wherein each of said plurality of baffles extends directly from said support, and wherein each of said plurality of baffles flexes to move relative to said first plate.

41. A MEMS flow module, as claimed in claim 36, wherein each of said plurality of baffles extend outwardly along a separate radii emanating from a common point.

42. A MEMS flow module, as claimed in claim 36, further comprising a flow restricting structure associated with each said first flow port, wherein each said flow-restricting structure is disposed about its corresponding said first flow port, wherein each said flow-restricting structure extends from one of said first plate and a corresponding said baffle and terminates prior to reaching the other of said first plate and said corresponding said baffle when said plurality of baffles are parallel with said first plate.

43. A MEMS flow module, as claimed in claim 36, wherein each said baffle comprises a plug that extends into, but is spaced from, its corresponding said first flow port.

44. A MEMS flow module, as claimed in claim 36, further comprising an annular support disposed about and spaced from said flow regulator, wherein said MEMS flow module further comprises at least one annular wall that interconnects said annular support and said first plate, and wherein said annular support and said plurality of baffles exist in a common fabrication level.

45. A MEMS flow module, as claimed in claim 36, wherein said plurality of baffles exist at least in a first fabrication level and said first plate exists at least in a second fabrication level that is spaced from said first fabrication level.

46. A MEMS flow module, as claimed in claim 36, further comprising a second plate that comprises a plurality of second flow ports and that is spaced from said first plate such said first plate is located between said plurality of baffles and said second plate, and wherein said MEMS flow module further comprises at least one structural interconnection extending between said first and second plates.

47. A MEMS flow module, as claimed in claim 46, wherein said plurality of baffles exist at least in a first fabrication level, wherein said first plate exists at least in a second fabrication level that is spaced from said first fabrication level, and wherein said second plate exists at least in a third fabrication level that is spaced from said second fabrication level such that said second fabrication level is located between said first and third fabrication levels.

48. A MEMS flow module, as claimed in claim 46, wherein said first plate comprises a plurality of first etch release holes, wherein said second plate comprises a plurality of second etch release holes, and wherein each of said plurality of baffles comprises a third etch release hole that is aligned with its corresponding said first flow port.

49. A MEMS flow module, as claimed in claim 48, further comprising a first flow restrictor for at least one said first etch release hole, a second flow restrictor for at least one said second etch release hole, and a third flow restrictor for at least one said third etch release hole.

50. A MEMS flow module, as claimed in claim 36, wherein said plurality of baffles are symmetrically disposed about a common point such that a length dimension of each said baffle is oriented so as to be other than along a radii extending from said common point, wherein corresponding portions of said plurality of baffles are equidistant from said common point.

51. A MEMS flow module, as claimed in claim 50, wherein each said baffle comprises a first point that is aligned with a center of its corresponding said first flow port, wherein each said baffle further comprises a second point that corresponds with a center of a region where said baffle is anchored, and wherein said first and second points are disposed at least generally the same distance from said common point.

52. A MEMS flow module, as claimed in claim 50, wherein each said baffle is individually anchored to said first plate.

53. A MEMS flow module, as claimed in claim 36, wherein said plurality of baffles are symmetrically disposed about a common point, wherein each baffle has a length dimension that extends along a first axis that fails to intersect with said common point, wherein said first axes of said plurality of baffles intersect so as to define an area, and wherein said common point is disposed within said area.

54. A MEMS flow module, as claimed in claim 36, wherein said first plate comprises a plurality of first etch release holes that extend through said first plate, wherein each of said plurality of baffles comprises at least one second etch release hole, and wherein each said second etch release hole extends through its corresponding said baffle.

55. A MEMS flow module, as claimed in claim 54, further comprising:
a first flow restrictor for at least one said first etch release hole and a second flow restrictor for at least one said second etch release hole for each said baffle.

56. An implant for addressing pressure within a first body region, comprising said MEMS flow module of claim 36 and a conduit, wherein said conduit comprises a flow path that is adapted to fluidly interconnect with the first body region, and wherein said MEMS flow module is disposed in said flow path.
